(19) United States
(12) Patent Application Publication
(10) Pub. No.: US 2010/0184060 A1
(43) Pub. Date: Jul. 22, 2010

(54) METHOD FOR THE IDENTIFICATION OF PROPANE-OXIDIZING BACTERIA

(75) Inventors: Francesco Rodriguez, San Donato Milanese (Milan) (IT); Francesca de Ferra, Lodi (IT); Elisabetta Franchi, Rozzano (Milan) (IT)

Correspondence Address: OBLON, SPIVAK, MCCLELLAND MAIER & NEUSTADT, L.L.P., 1940 DUKE STREET, ALEXANDRIA, VA, 22314, US

(73) Assignee: ENI S.p.A., Rome (IT)

(21) Appl. No.: 12/666,067

(22) PCT Filed: Jun. 10, 2008
(86) PCT No.: PCT/EP2008/004723

§ 371 Date: Mar. 25, 2010

(30) Foreign Application Priority Data
Jun. 22, 2007 (IT) MI2007A001262

(51) Int. Cl. C12Q 1/68; C07H 21/04

(52) U.S. Cl. 435/6; 536/23.2; 536/23.7; 536/24.33

(57) ABSTRACT

The invention relates to a method for the identification of propane-oxidizing bacteria which is based on the identification of at least one fragment of the prmA gene encoding the alpha subunit of the propane monooxygenase enzyme and/or the prmD gene encoding an ancillary protein involved in the oxidation reaction of propane by gene amplification in the presence of pairs of primers selected in correspondence of homologous portions, deduced from the alignment of the prmA and prmD sequences.

The present invention relates to a method for the identification of propane-oxidizing bacteria in environmental samples.

More specifically, the present invention relates to a method for the identification of propane-oxidizing bacteria which is based on the use of specific probes for this group of bacteria.

The method of the invention can be used in oil search which is based on surface analysis techniques (Surface geochemical Exploration) and allows the presence of oil or natural gas reservoirs to be identified in the underlying area.

It is known that, in many cases, oil and gas reservoirs are not watertight and a certain quantity of more or less volatile molecules can reach the surface migrating across the porosity of the rocks as far as the ground surface.

This release (seepage or seep) can be macroscopically visible in accumulation areas: in this case the phenomenon is defined macroseepage (macroseep). Macroseeps are generally localized at the end of faults or fractures.

In other cases, the seepage concerns a reduced quantity of short-chain hydrocarbons, in the gaseous state; these traces can only be revealed with specific analyses: in this case it is a microseepage (microseep) [Schumacher D., Abrams M. A. eds., 1996, Hydrocarbon Migration and its Near-Surface Expression, AAPG Memoir 66, 445 p].

Between the two extremes, there can be intermediate manifestations depending on the characteristics of the reservoir itself and the geological characteristics of the overlying stratum. The seepages are visible both on-shore and off-shore.

In oil search based on surface analysis techniques (Surface Geochemical Exploration) particular attention is paid to microseeps, as the gaseous hydrocarbons can migrate, also with not well-defined mechanisms, vertically above the reservoirs, allowing them to be localized [Saunders, D. F., Burson K. R., Thompson C. K., Model for Hydrocarbon Microseepage and Related Near-Surface Alterations, AAPG Bulletin, V83 Nr. 1 (January 1999), p 170-185; Nunn J., A., Meulbroek, P., Kilometer-scale upward migration of hydrocarbons in geopressured sediments by buoyancy-driven propagation of methane-filled fractures, AAPG Bulletin, V86 Nr. 5 (May 2002), p 907-918].

Various explorative technologies are grouped under the name of “surface geochemical exploration”, which allow the presence of hydrocarbons or the effects produced by their presence (anomalies) to be directly or indirectly identified.

The anomalies produced can be of the physico-chemical or biological type. An anomaly found in areas overlying a reservoir is revealed by the appearance of bacterial populations able to use the hydrocarbons coming from the sub-surface as carbon source for their growth; among these, for example, various species able to oxidize methane have been characterized; as methane is a molecule which is widely diffused in the environment and produced biologically, these bacterial systems are less important for the present purpose.

Bacteria which oxidize propane and use it for their metabolism are of greater interest, as this molecule is not produced biologically: propane is normally present at the level of microseeps together with methane, ethane, butane and other short-chain alkanes (gaseous or extremely volatile).

The detection of the presence of propane-oxidizing bacteria can be carried out through microbiological methods which essentially derive from two fundamental techniques: MPOG (Microbial Prospection for Oil and Gas) and MOST (Microbial Oil Survey Technique). During the microbiological surveys, samples of soil are collected at 20-150 cm below the surface (both onshore and offshore); the bacterial cells are cultivated in the laboratory using the molecules typically identified in microseeps as carbon sources; under normal conditions, the microbial populations need to induce the enzymatic pool for the oxidation of the specific substrate and there is therefore a certain time lapse (lag) between inoculum and growth; cells which already grow in an environment in which the molecule is present, on the contrary, do not need any adaptation period, and growth is therefore relatively immediate. In relation to the consistency of the populations, the duration of the lag and other biochemical parameters, it is possible to assume the presence of a gas source beneath the collection area [Wagner, M., M. Wagner, J. Piske, R. Smit (2002), Case Histories of Microbial Prospection for Oil and Gas, Onshore and Offshore in Northwest Europe—in: Surface exploration case histories: Applications of geochemistry, magnetics, and remote sensing, D. Schumacher and L. A. LeSchack eds., AAPG Studies in Geology No. 45 and SEG Geophysical References Series Nr. 11, p 453-479].

The main disadvantage in the use of this technology is represented by the fact that the cultivation of these bacterial strains on specific culture mediums is slow or very slow; it is also known that only a minimum part of the microbial species can be cultivated under normal laboratory conditions and, in addition, the behaviour of the populations examined can vary considerably giving results which are difficult to standardize.

Although cultivation methods are continually evolving [Green, B. D. and Keller, M., Capturing the uncultivated majority, Current Opinion in Biotechnology 2006, 17:1-5], biomolecular techniques have proved under various circumstances to be more suitable for characterizing bacterial populations in their habitat. Genes with specific activities of interest, for example, can be identified in environmental samples with standard techniques such as PCR (Polymerase Chain Reaction) with the use of probes ad hoc designed on identical sequences or with different degrees of homology. It is also possible, with correlated techniques, to both quantify the genes themselves and their transcription products (mRNA).

The quantification of the genes can be performed by means of techniques such as qPCR (quantitative PCR) whereby it is possible to obtain the amount of specific gene in a sample of soil by previously constructing a standard calibration curve at a known concentration. By applying qPCR to the quantification of the RNA messenger, by using the technique called RT PCR, it is possible to obtain informations about the level of activity of the gene which is most correlated with the quantity of propane effectively present: this represents an indirect measurement of the quantity of propane which reaches the surface from the reservoir and therefore allows the underlying reservoir to be identified.

A method has now been found, based on the amplification of specific genes encoding for a family of propane monooxygenase, that allows to identify bacterial populations which use propane. These enzymes are responsible for the first reaction which enable the use of propane as carbon source: the oxidation of propane to propanol.

An object of the present invention therefore relates to DNA sequences deduced from the chromosomal DNA of propane-oxidizing bacteria, comprising the gene prmA encoding the alpha subunit of the propane monooxygenase enzyme, characterized by the nucleotide sequences indicated in Table 4.

A further object of the present invention relates to DNA sequences deduced from the chromosomal DNA of propane-oxidizing bacteria, comprising the gene prmD encoding an ancillary protein involved in the oxidation reaction of propane, characterized by the nucleotide sequences indicated in Table 5.

Another object of the present invention relates to a method for the identification of propane-oxidizing bacteria comprising the extraction of DNA from environmental samples and the subsequent identification of at least one fragment of the gene prmA, or of the gene pimp, characterized in that the identification of the gene fragment is carried out by gene amplification in the presence of primers selected in correspondence of homologous portions deduced from the alignment of the prmA and prmD sequences indicated above.

In particular, the identification of the gene prmA can be effectively carried out by gene amplification in the presence of combinations of selected primers or derivatives by partial degeneration from the following groups:

FORWARD PRIMERS for prmA:
(SEQ ID NO: 1)
prmA_1F: CTTCCCGATGGARGARGARAARGA
(SEQ ID NO: 2)
XA_0301F: GCCCATGCGAAGATCACCGA
(SEQ ID NO: 3)
XA_0358F: CCGCTTCGGCACCGACTACAC
(SEQ ID NO: 4)
XA_0370F: ACCGACTACACCTTCGAGAAGGC
(SEQ ID NO: 5)
XA_0382F: TTCGAGAAGGCCCCCAAGAAGGA
(SEQ ID NO: 6)
XA_0406F: CCTCTCAAGCAGATCATGCGGTC
(SEQ ID NO: 7)
XA_0930F: ACGGTCTTCCACTCGGTGCAGTC
(SEQ ID NO: 8)
XA_0993F: TGATGGCGCTCGCCGACGAGCG
(SEQ ID NO: 9)
XA_1041F: CTGCGGTACGCGTGGTGGAACAA
(SEQ ID NO: 10)
XA_1089F: GCACCTTCATCGAGTACGGCAC
(SEQ ID NO: 11)
XA_1107F: CGGCACCAAGGACCGCCGCAAGGA
(SEQ ID NO: 12)
XA_1152F: GGCGGCGGTGGATCTACGACGA
(SEQ ID NO: 13)
XA_1170F: TCATCCCGCTCGAGAAGTACGG
(SEQ ID NO: 14)
XA_1233F: GTCGAGGAGGCGTGGAAGCG
(SEQ ID NO: 15)
XA_1305F: GGCTGGCCGGTGAACTACTGGCG
(SEQ ID NO: 16)
XA_1390F: TCCAAGTACGGCAAGTGGTGGGAG
(SEQ ID NO: 17)
XA_1485F: ACCGGTGCTGGACCTGCATGGT
(SEQ ID NO: 18)
XA_1625F: GGCCGCCCGACCCCGAACATGGG
(SEQ ID NO: 19)
XA_460F: GTGTACGGCGCCATGGACGG
(SEQ ID NO: 20)
XA_526F: CTCGAATGGCAGAAGCTGTTCCT
(SEQ ID NO: 21)
XA_586F: GCGATGCCGATGGCCATCGACGC
(SEQ ID NO: 22)
XA_745F: AAGGCGTTCGCGAACAACTACGC
(SEQ ID NO: 23)
XA_789F: TTCGGTGAAGGCTTCATCACCGG
(SEQ ID NO: 24)
prmA_2F: GGTCGCCGAGACNGCNTTYACNAA
(SEQ ID NO: 25)
prmA_49F: GCGAAGATCACCGAGCTGT
(SEQ ID NO: 26)
prmA_733 (f): CGCAATCGTCCGCTGCTC
(SEQ ID NO: 27)
XA_16F: GGCGCACATTGAGTAGGCA
(SEQ ID NO: 28)
XA_17F: TGCAGATGATCGACGAGGT
(SEQ ID NO: 29)
XA_18F: TCGCGGCACATCTCCAACGG
(SEQ ID NO: 30)
XA_19F: CGGACTTCGAGTGGTTCGA
(SEQ ID NO: 31)
XA_20Rf: AACAAGCCGATCGCGTTCG
(SEQ ID NO: 32)
XA_21Rf: CCGAACATGGGCCGGCTCA
(SEQ ID NO: 33)
XA_22F: GCCCGACCCCGAACATGGG
(SEQ ID NO: 34)
XA_23Rf: TGGCAGAAGCTGTTCCTGTCGAT
(SEQ ID NO: 35)
XA_24F: AGCTACGCCGAGATGTGGC
(SEQ ID NO: 36)
XA_25Rf: TGGATCTACGACGACTACTAC
(SEQ ID NO: 37)
XA_26F: GTCCGCGACGACGGCAAGACC
(SEQ ID NO: 38)
XA_27Rf: AAGCAGATCATGCGGTCCTAC
(SEQ ID NO: 39)
XA_28F: GTCCGCGACGACGGCAAGAC
(SEQ ID NO: 40)
XA_29F: TCCGCGGCAACATGTTCCG
(SEQ ID NO: 41)
XA_30F: GCGGTGCAGATGATCGACGA
(SEQ ID NO: 42)
XA_31Rf: GAGATGTGGCGGCGGTGGA
(SEQ ID NO: 43)
XA_32Rf: AACTACTGGCGGATCGACGCG
(SEQ ID NO: 44)
XA_33Rf: GACGGCAAGACCCTGGTC
(SEQ ID NO: 45)
Xmo_10F: TGGTGGAACAACCACTGCGTGGT
(SEQ ID NO: 46)
Xmo_11F: CAGTGGCGGACCTACTGCTCGG
(SEQ ID NO: 47)
Xmo_1F: TGGTTCGAGCACAACTAYCCNGGNTGG
(SEQ ID NO: 48)
Xmo_3Rf: AAGCCGATCGCGTTCGAGGA
(SEQ ID NO: 49)
Xmo_4F: GATACCAGTACCCGCACCG
(SEQ ID NO: 50)
Xmo_5Rf: CAGATGAACCTCAAGAAGCT
(SEQ ID NO: 51)
Xmo_6F: TACATGAACAACTACATCGA
(SEQ ID NO: 52)
Xmo_9F: CAGGAGGCGCACATTGAGTAGG
(SEQ ID NO: 53)
Xmo_F: ACGATCCAGATGAACCTCAAGA
(SEQ ID NO: 54)
Xmo_Rf: TACGCCGAGATGTGGCGGC

REVERSE PRIMERS for prmA:

(SEQ ID NO: 55)

XA_30Fr: ACCTCGTCGATCATCTGCA

(SEQ ID NO: 56)

XA_0288R: GACAACTCGGTGATCTTCGC

(SEQ ID NO: 57)

XA_0348R: GCCTTCTCGAAGGTGTAGTCGGT

(SEQ ID NO: 58)

XA_0360R: TCCTTCTTGGGGGCCTTCTCGAA

(SEQ ID NO: 59)

XA_0393R: CGGGAAGTAGGACCGCATGATCTG

(SEQ ID NO: 60)

XA_0408R: TTCTCTTCCTCCATCGGGAAGTA

(SEQ ID NO: 61)

XA_0444R: GGCACCGTCCATGGCGCCGTA

(SEQ ID NO: 62)

XA_0567R: ACCGCGTCGATGGCCATCGGCAT

(SEQ ID NO: 63)

XA_0624R: TGACGAACCTCGTCGATCATCTG

(SEQ ID NO: 64)

XA_0745R: CCGATGGTGCCCGCGTAGTTGTT

(SEQ ID NO: 65)

XA_0779R: GGTGATCGCGTCGCCGGTAATGAA

(SEQ ID NO: 66)

XA_0866R: TTGGCGGCCGCCTCGTCGGGCAT

(SEQ ID NO: 67)

XA_0944R: GAGTAGCCGTTGGAGATGTG

(SEQ ID NO: 68)

XA_0983R: AGTGGACGGTTGCGCTCGTCGGC

(SEQ ID NO: 69)

XA_1073R: TCCTTGGTGCCGTACTCGATGAA

(SEQ ID NO: 70)

XA_1091R: TCCCGGTCCTTGCGGCGGTCCTT

(SEQ ID NO: 71)

XA_1214R: CGCTTCCACGCCTCCTCGAC

(SEQ ID NO: 72)

XA_1327R: TGTGCTCGAACCACTCGAAGTCC

(SEQ ID NO: 73)

XA_1469R: GCGGGAACCATGCAGGTCCAGCA

(SEQ ID NO: 74)

XA_1548R: GTCCAGTAGCAGGTTTCCGAGCA

(SEQ ID NO: 75)

XA_1615R: CCCGTGAGCCGGCCCATGTTCGG

(SEQ ID NO: 76)

XA_1714R: TGACCGACCAGGGTCTTGCCGTC

(SEQ ID NO: 77)

XA_18Fr: CCGTTGGAGATGTGCCGCGA

(SEQ ID NO: 78)

XA_19Fr: TCGAACCACTCGAAGTCCG

(SEQ ID NO: 79)

XA_20R: CGAACGCGATCGGCTTGTT

(SEQ ID NO: 80)

XA_21R: TGAGCCGGCCCATGTTCGG

(SEQ ID NO: 81)

XA_22Fr: CCCATGTTCGGGGTCGGGC

(SEQ ID NO: 82)

XA_23R: ATCGACAGGAACAGCTTCTGCCA

(SEQ ID NO: 83)

XA_24Fr: GCCACATCTCGGCGTAGCT

(SEQ ID NO: 84)

XA_25R: GTAGTAGTCGTCGTAGATCCA

(SEQ ID NO: 85)

XA_26Fr: GGTCTTGCCGTCGTCGCGGAC

(SEQ ID NO: 86)

XA_27R: GTAGGACCGCATGATCTGCTT

(SEQ ID NO: 87)

XA_28Fr: GTCTTGCCGTCGTCGCGGAC

(SEQ ID NO: 88)

XA_29Fr: CGGAACATGTTGCCGCGGA

(SEQ ID NO: 89)

XA_30Fr: TCGTCGATCATCTGCACCGC

(SEQ ID NO: 90)

XA_31R: TCCACCGCCGCCACATCTC

(SEQ ID NO: 91)

XA_32R: CGCGTCGATCCGCCAGTAGTT

(SEQ ID NO: 92)

XA_33R: GACCAGGGTCTTGCCGTC

(SEQ ID NO: 93)

Xmo_10R: ACCACGAGTAGGTCCGCCACTG

(SEQ ID NO: 94)

Xmo_11R: CCGAGCAGTAGGTCCGCCACTG

(SEQ ID NO: 95)

Xmo_2R: TGCGGCTGCGCGATCAGCGTYTTNCCRTC

(SEQ ID NO: 96)

Xmo_3R: TCCTCGAACGCGATCGGCTT

(SEQ ID NO: 97)

Xmo_4Fr: CGGTGCGGGTACTGGTATC

(SEQ ID NO: 98)

Xmo_5R: AGCTTCTTGAGGTTCATCTG

(SEQ ID NO: 99)

Xmo_6Fr: TCGATGTAGTTGTTCATGTA

(SEQ ID NO: 100)

Xmo_Fr: TCTTGAGGTTCATCTGGATCGT

(SEQ ID NO: 101)

Xmo_R: GCCGCCACATCTCGGCGTA.

The identification of the prmD gene can be effectively carried out by means of gene amplification in the presence of combinations of selected primers (or derivatives by partial degeneration) from the following groups:

| | |
|---|---|
| FORWARD PRIMERS for prmD: | |
| XD_043F: TCGTCCACCGAGTTCTCCAACA | (SEQ ID NO: 102) |
| XD_071F: GTGTCACCTTGATGAACACCCC | (SEQ ID NO: 103) |
| XD_181F: AACCGGCTCGAGTTCGACTACG | (SEQ ID NO: 104) |
| XD_2Rf: GTTCTCCAACATGTGCGGCG | (SEQ ID NO: 105) |
| XD_3Rf: CCGTCGATGATCCGCGTC | (SEQ ID NO: 106) |
| XD_4Rf: TCTTCGAGGAGATCAGCTCCAC | (SEQ ID NO: 107) |
| XD_5Rf: GACGCCGCCGAGTACATCGG | (SEQ ID NO: 108) |
| Xmo_8F: ACCGAGTTCTCCAACATGTG | (SEQ ID NO: 109) |
| XD_6Rf: TTCGAGGAGATCAGCTCCACC | (SEQ ID NO: 110) |
| Xmo_7Rf: CATGCAATTCGGATCGKCCA | (SEQ ID NO: 111) |
| XD_7F: GGCTCCATCTTCGAGGAGATCA | (SEQ ID NO: 112) |

| | |
|---|---|
| REVERSE PRIMERS for prmD: | |
| prmD_1R: ATGGACCATCCGNCCRTARTGNGT | (SEQ ID NO: 113) |
| XD_061R: ACGCGGCCGATCGGGGTGTTCAT | (SEQ ID NO: 114) |
| XD_136R: TGGCCGTCGACGCGGATCATCGA | (SEQ ID NO: 115) |
| XD_172R: TCGGTGAGCTCGTCGTAGTCGAA | (SEQ ID NO: 116) |
| XD_235R: TGGGTGGAGCTGATCTCCTCGAA | (SEQ ID NO: 117) |
| XD_2R: CGCCGCACATGTTGGAGAAC | (SEQ ID NO: 118) |
| XD_3R: GACGCGGATCATCGACGG | (SEQ ID NO: 119) |
| XD_4R: GTGGAGCTGATCTCCTCGAAGA | (SEQ ID NO: 120) |
| XD_5R: CCGATGTACTCGGCGGCGTC | (SEQ ID NO: 121) |
| XD_6R: GGTGGAGCTGATCTCCTCGAA | (SEQ ID NO: 122) |
| XD_7Fr: TGATCTCCTCGAAGATGGAGCC | (SEQ ID NO: 123) |
| Xmo_7R: TGGMCGATCCGAATTGCATG | (SEQ ID NO: 124) |
| Xmo_8Fr: CACATGTTGGAGAACTCGGT. | (SEQ ID NO: 125) |

The sequences of the primers of the invention were first deduced from the alignment of genes encoding the subunits of the enzymatic systems homologous to propane monooxygenases belonging to the family of the “soluble diirron monooxygenases” responsible for the oxidation of alkanes, alkenes and similar short-chain molecules [Leahy J. G., Batchelor P. J., Morcomb S. M., Evolution of the soluble diiron monooxygenases, FEMS Microbiology Reviews 27 (2003) 449-479].

The sequences were aligned with the use of Clustal X software [Thompson, J. D., Higgins, D. G. and Gibson, T. J. (1994) CLUSTAL W: Improving the sensitivity of progressive multiple sequence alignment through sequence weighting, positions-specific gap penalties and weight matrix choice, Nucleic Acids Research, 22:4673-4680]), in order to define the kept regions and identify, in homologous areas, the specific nucleotide sequences to be used as primers for the amplification of homologous genes present in strains isolated from environmental samples.

On the basis of the information obtained from the sequencing and alignment of the sequences of said genes or from their gene product, the primers of the present invention were subsequently constructed.

The method of the invention revealed a greater sensitivity, specificity and rapidity with respect to the methods described in the known art (MPOG, MOST).

A further object of the present invention relates to oligonucleotides having a sequence selected from those indicated above.

These oligonucleotides, as all oligonucleotides deriving from the prmA and prmD sequences identified in Tables 4 and 5, cannot only be used as primers for gene amplification but also as gene probes for the identification of the prmA gene and prmD gene of propane-oxidizing bacteria.

In this case, by using techniques of the known art, the oligonucleotides of the invention or fragments of the prmA or prmD genes, amplified or cloned or synthesized, are subjected to labelling so that they can be easily detected and subsequently subjected to hybridization with the genomic DNA to be analyzed [as for example in the FISH technique (fluorescence in situ hybridization)] which allows specific sequences to be identified by fluorescence in samples containing bacterial cells as described for example in “In Situ Hybridization. A practical Approach” Edited by D. G. Wilkinson IRL Press, Oxford University Press, 1994.

The labelling can be carried out with various techniques such as, for example, fluorescence, radioactivity, chemiluminescence or enzymatic labelling.

The detection method of propane-oxidizing bacteria of the invention comprises, in particular, the following actions:

extracting the DNA from samples;

putting the extracted DNA in contact with a pair of primers selected from oligonucleotides having the sequences previously indicated, under conditions allowing the specific amplification of a fragment of the prmA or prmD gene [or alternatively using other analysis methods such as quantitative PCR, qPCR (Dorak M. T. (ed.), Real-time PCR, Taylor & Francis (2006)].

analyzing the gene amplification product by means of gel-electrophoresis.

The sample to be analyzed may consist of soil or water coming from environmental samples or from bacterial cultures.

The extraction of genomic DNA from the samples to be analyzed can be carried out according to standard techniques or with the use of commercial kits.

These techniques, associated with the rapidity of the analysis with the primers, object of the invention, considerably reduce the detection times of propane-oxidizing bacteria, allowing them to be detected and quantified within a few hours; the methods commonly used, on the other hand, which are based on the effective bacterial cultivability, require much longer times: at least a week.

A pair of oligonucleotides having a sequence essentially identical to or comprising those previously indicated or deriving from other homologous portions of the sequences of the prmA or prmD genes, are used as primers for the amplification.

“Essentially identical” means that the sequence of oligonucleotides is essentially identical to those previously identified or that it is different from these without influencing their capacity of hybridizing with the prmA or prmD gene.

The gene amplification method used is based on the reaction of a DNA polymerase in the presence of a pair of primers and is well known to experts in the field (Sambrook et al., 1989, Molecular Cloning, Cold Spring Harbor, N.Y.).

“Conditions which allow gene amplification” refer to temperature conditions, reaction times and, optionally, additional agents which are necessary for allowing the fragment of the prmA or prmD genes to be recognized by the primers of the invention and copied identically.

“Conditions which allow specific amplification” refer to conditions which prevent the amplification of sequences different from those of the prmA or prmD genes.

According to the method of the invention, the “pairing” step during the amplification reaction is carried out at temperatures compatible with the sequence of the primers, preferably, in this specific case, at 58° C.

The buffers and the enzymes used are solutions compatible with the characteristics of the DNA polymerases used, such as for example Taq polymerases, ampliTaq Gold and hot-start polymerases, polymerases from hyperthermophile microorganisms.

Polymerases such as Taq polymerases are preferably used in the presence of the buffer solution most appropriate for the type of enzyme.

The sequences corresponding to the pairs of primers identified by the present invention have produced particularly interesting results in the quantitative determination of propane-oxidizing bacteria.

A further advantage of the method described is the easiness of adaptation to protocols to be used “in situ” such as for example the use of portable real-time PCR instruments.

The following examples and figures illustrate the invention without limiting its scope.

### EXAMPLE 1

### Isolation of Propane-Oxidizing Strains

Samples of soil overlying known oil reservoirs were recovered; 0.2-1 gr of each sample were resuspended in 10 ml of minimal culture medium without a carbon source and incubated overnight under stirring at 20-25° C.

### Minimal Medium (Per Litre):

### Kh₂PO₄: 5 g

### NH₄Cl: 1.25 g

NaOH: up to pH=7.4

### MgSO₄: 0.2 gr

### CaCl₂: 26 mg

### FeCl₃: 10 mg

### MnCl₂: 2.5 mg

### ZnCl₂: 1.5 mg

### CuCl₂: 0.5 mg

### CoCl₂: 0.5 mg

### Na₂MoO₄: 0.5 mg

### NiCl₂: 0.15 mg

### H₃BO₃: 1.5 mg

### Na₂O₃Se: 0.1 mg

After decanting the suspensions, 0.1-1 ml aliquots were incubated in a minimal medium in the presence of propane or, alternatively, of a mixture of normal- and 2-propanol (0.2% final for each); the cultures in propanol were subjected to an enrichment period of three days at 25° C. before being diluted, at least 1:100, in the same medium but in the presence of propane as carbon source. The step in the presence of alcohols as carbon source is not indispensable, but it allows to speed up the enrichment process; if the process continues for too long times there is a prevalence of Pseudomonas (generally unable to oxidize propane).

Once transferred in the presence of propane, the cells were incubated until the cultures showed an evident turbidity; aliquots were then streaked on solid medium containing the mixture of alcohols as carbon source; after the growth of the colonies, these were inoculated individually into minimal medium in the presence of propane as carbon source. When the growth was complete, aliquots of the culture were streaked again on both plates of minimal medium in the presence of the mixture of alcohols and on plates of rich medium (LB) for further characterization and to verify the purity of the cultures before further experiments and before keeping in the form of glycerinates. A single colony per morphological type was streaked from each plate (at this stage pure cultures are generally obtained and consequently there is a single morphologic type per plate).

### EXAMPLE 2

### Characterization of Propane-Oxidizing Strains

The colonies were characterized from a taxonomical point of view by amplification of a portion of 16S rDNA and subsequent sequencing.

For the purification of the genomic DNA, the strains were inoculated in 10 ml of rich medium (typically 10 gr/1 of Peptone, 5 gr/1 of Yeast Extract and 5 gr/1 of NaCl) and incubated at 28.5° C. for 2-3 days, until an evident turbidity is obtained.

The cells were collected by centrifugation and resuspended in 950 μl of TE (10 mM Tris/Cl, 1 mM EDTA, pH 8) in the presence of lysozyme (1 mg/ml). After incubating the suspensions for 20′ at 37° C., 50 μl of 10% SDS and 5 μl of a solution containing protease K (stock 20 mg/ml), were added.

The samples were incubated for 1 h at 37° C.; 100 μl of 3 M K acetate, pH 5, were then added and the mixture was incubated in ice for 10′; after centrifuging for 15′ at 4° C. at 20800 RCF, the DNA was precipitated from the supernatant by the addition of one volume of isopropanol and by centrifugation as before. The precipitate was washed in 70% ethanol, dried and dissolved in 800 μl of TE in the presence of 20 μg of Ribonuclease A (pancreatic). The samples were extracted with one volume of a mixture of phenol/chloroform/isoamyl alcohol (25:24:1) and subsequently with one volume of a mixture of chloroform/isoamyl alcohol. The DNA was finally precipitated with one volume of 2-propanol after the addition of 0.1 volumes of 3M K acetate, pH 5; after washing the pellet with 70% ethanol, the DNA was dissolved in H₂O at a concentration equal to about 50 ng/μl.

The genomic DNA was amplified with the pair of primers Rho_{—}1F and Rho_{—}4R or Rho_{—}1F and Rho_{—}9R shown in Table 1.

All the primers whose sequence is indicated in Table 1 were used for the sequencing.

The primers sequences are obtained from the alignment of rDNA 16S sequences deposited at the National Center for Biotechnology Information (http://www-ncbi.nlm.nih.gov/). The alignments were carried out by grouping the sequences into classes using the clustalW program [Thompson, J. D., Higgins, D. G. and Gibson T. J. (1994) CLUSTAL W: improving the sensitivity of progressive multiple sequence alignment through sequence weighting, positions-specific gap penalties and weight matrix choice. Nucleic Acids Research, 22:4673-4680] as implemented within the BioEdit software [Hall, T. A. 1999. BioEdit: a user-friendly biological sequence alignment editor and analysis program for Windows 95/98/NT. Nucl. Acids. Symp. Ser. 41:95-98]: those presented in the Table, proved to be the best combination for the strains isolated, were obtained by aligning the sequences belonging to the Actinobacteria class.

About 5 ng of genomic DNA in final 20 μl for each sample, were used for the PCRs; the dNTPs were mixed at a concentration equal to 200 μM each; the primers were used at a concentration of 0.5-1 pmole/μl of reaction mixture; the enzyme, Taq polymerase (New England Biolabs), was added to a final concentration of 2.5 U for every 100 μl of reaction mixture.

After an initial step at 95° C. for 2′, 7 cycles were carried out with an initial denaturation for 30″ at 94° C., a pairing step for 30″ at 62° C. reducing the temperature by 1° C. for each cycle to 56° C. and an elongation for 1′30″ at a temperature of 72° C.; 35 cycles were added to these with an initial denaturation at 94° C. for 30″, a pairing step for 30″ at 58° C. and a polymerization for 1′30″ at 72° C.

4 μl of each sample, obtained by amplification, to which 1 μl of ExoSAP-IT (USB) was added, were used for the sequencing; after an incubation for 30′ at 37° C., the samples were incubated at a denaturation temperature of 90° C. for 10′ to neutralize the activity of the enzymes. 3 pmoles of specific primer were added to each sample, in the presence of 1 μl of reaction mixture (DYEnamic ET Terminator Cycle Sequencing Kit, Amersham). After a step at 95° C. for 1′, 30 of the following cycles were carried out to promote the sequencing reaction: 30″ at 94° C., 30″ at 56° C. and 2′ at 60° C.

The sequences obtained were compared with those present in the data banks at the National Center for Biotechnology Information (http://www-ncbi.nlm.nih.gov/BLAST/) using the “blast” program [Altschul, S. F., Gish, W., Miller, W., Myers, E. W. & Lipman, D. J. (1990) “Basic local alignment search tool”. J. Mol. Biol. 215:403-410].

From an analysis of the alignments produced it can be seen that the strains selected belong to the Rhodococcus, Gordonia and Mycobacterium genus:

SMV048: Gordonia sp.

SMV049: Rhodococcus sp.

SMV052: Rhodococcus sp.

SMV105: Rhodococcus sp.

SMV106: Rhodococcus sp.

SMV152: Rhodococcus sp.

SMV153: Rhodococcus sp.

SMV154: Rhodococcus sp.

SMV155: Rhodococcus sp.

SMV156: Rhodococcus sp.

SMV157: Rhodococcus sp.

SMV158: Mycobacterium sp.

SMV160: Rhodococcus sp.

SMV161: Rhodococcus sp.

SMV162: Rhodococcus sp.

SMV163: Gordonia sp.

SMV164: Rhodococcus sp.

SMV167: Rhodococcus sp.

SMV168: Rhodococcus sp.

SMV169: Rhodococcus sp.

SMV170: Rhodococcus sp.

SMV171: Rhodococcus sp.

SMV172: Rhodococcus sp.

SMV173: Rhodococcus sp.

SMV174: Rhodococcus sp.

### EXAMPLE 3

### Identification of the Sequences Encoding Propane Monooxygenases

Some of the enzymes able to oxidize gaseous alkanes (such as methane, propane and butane) and short-chain alkenes, linear or branched, belong to the group of the so-called “Soluble Diiron Monooxygenases”. These are enzymes consisting of various subunits which catalyze the first reaction, in which the alkane is oxidized to primary or secondary alcohol, the alpha subunit of which contains the catalytic site [Leahy J. G., Batchelor P. J., Morcomb S. M., Evolution of the soluble diiron monooxygenases, FEMS Microbiology Reviews 27 (2003) 449-479].

By aligning the known sequences of the different subunits, it was possible to identify various subgroups such as, for example, Methane Monooxygenases of the soluble type (sMMO), butane monooxygenases, alkene monooxygenases and monooxygenases more specific for aromatic compounds [F. Rodriguez, E. Franchi, L. P. Serbolisca, F. de Ferra. Monitoring of Bacterial Species Involved in Light Hydrocarbon Oxidation from Oil Reservoirs to the Surface. The Joint International Symposia for Subsurface Microbiology (ISSM 2005) and Environmental Biogeochemistry (ISEB XVII) Jackson Hole, Wyo.—Aug. 14-19, 2005]; this allowed to select a group of monooxygenases, more homologous with each other, able to oxidize molecules chemically related to propane: the only monooxygenase known for being capable of oxidizing propane, also belongs to this group [Kotani T., Yamamoto T., Yurimoto H., Sakai Y., Kato, N., Propane monooxygenase and NAD+-dependent secondary alcohol dehydrogenase in propane metabolism by Gordonia sp. strain TY-5, J. Bacteriol. 185 (24), 7120-7128 (2003)] and a monooxygenase from Frankia sp. Cc13 (Acc. Num. AAIE01000085) which has an extremely high homology, deposited as methane monooxygenase [Copeland, A., Lucas, S., Lapidus, A., Barry, K., Detter, C., Glavina, T., Hammon, N., Israni, S., Pitluck, S and Richardson, P., US DOE Joint Genome Institute (JGI-PGF), Sequencing of the draft genome and assembly of Frankia sp. Cc13].

The subunits of these enzymatic complexes are encoded at the level of operons in which the order of the single genes is maintained: A, B, C, D followed by two genes with a not well known function, the gene for a alcohol dehydrogenase (adh) and that for a chaperonine (GroEL).

Some portions with a greater homology were selected from the alignment of the amino-acidic sequences of the alpha subunit, which in Gordonia sp. TY-5 is encoded by prmA, as indicated in Table 2.

The following two degenerated oligonucleotides used in the first amplification experiments were obtained from the sequences in Table 2:

(SEQ ID NO: 47)

Xmo_1F: TGGTTCGAGCACAACTAYCCNGGNTGG

(SEQ ID NO: 95)

Xmo_2R: TGCGGCTGCGCGATCAGCGTYTTNCCRTC

N indicates any nucleotide, Y indicates C or T and R indicates A or G.

A portion with a greater homology with the sequence indicated in Table 3 was also selected from the alignment of the amino-acid sequences of the subunits encoded by prmD.

The primer with the following sequence was obtained from the amino-acid sequence:

| | |
|---|---|
| prmD_1R: ATGGACCATCCGNCCRTARTGNGT | (SEQ ID NO: 113) |

N indicates any nucleotide whereas R indicates A or G. The partial sequencing of the prmD gene was initially carried out on an amplification product obtained using the primer prmD_{—}1R combined with the primer Xmo_{—}6F deduced from the prmA sequences:

### Xmo_{—}6F: TACATGAACAACTACATCGA (SEQ ID NO:51)

Similarly, a partial sequence of the prmB gene was obtained for some strains using the primers mapping in the final portion (3′) of prmA; these sequencing experiments were initially carried out on the amplification products previously mentioned and also after inverse amplification; in particular, the primers XA_{—}22F, XA_{—}26F and XA_{—}28F listed in the section “FORWARD PRIMERS for prmA” were used for the initial sequencing.

Portions of these genes from strains isolated from environmental samples and selected for their ability to grow on propane as the sole carbon source, were amplified and sequenced with the primers indicated above.

The sequencing was carried out on both direct amplification products and inverse amplification and “primer walking” to lengthen the sequences known from each previous experiment. New-generation oligonucleotides were designated from the alignments of the partial sequences; the sequences of these primers are indicated in the lists provided above: FORWARD PRIMERS for prmA”, REVERSE PRIMERS for prmA”, “FORWARD PRIMERS for prmD” and “REVERSE PRIMERS for prmD”.

These primers allowed to complete the sequence of genes A and D from the strains isolated from the environmental samples previously mentioned (Gordonia sp. SMV048, Rhodococcus sp. SMV049, 052, 105, 106, 152, 153, 154, 155, 156, 157, Mycobacterium SMV158, Rhodococcus sp. SMV 160, 161, 162, Gordonia SMV163 and Rhodococcus sp. SMV164, 167, 168, 169, 170, 171, 172, 173 and 174). The sequences relating to prmA and prmD are indicated in Table 4 and Table 5.

### EXAMPLE 4

### Amplification of the Genes prmA from Genomic DNA of Isolated Bacterial Strains

Different “universal” primers can be designed from known sequences, allowing the amplification of portions of the genes prmA from both purified strains and environmental samples.

Some of the pairs of primers which can be conveniently used for the amplification of the prmA genes are the following:

| | |
|---|---|
| XA_16F: GGCGCACATTGAGTAGGCA | (SEQ ID NO: 27) |
| XA_23R: ATCGACAGGAACAGCTTCTGCCA | (SEQ ID NO: 82) |
| or | |
| XA_16F: GGCGCACATTGAGTAGGCA | (SEQ ID NO: 27) |
| Xmo_5R: AGCTTCTTGAGGTTCATCTG | (SEQ ID NO: 98) |
| or | |
| XA_19F CGGACTTCGAGTGGTTCGA | (SEQ ID NO: 30) |
| XA_21R TGAGCCGGCCCATGTTCGG | (SEQ ID NO: 80) |

About 5 ng of genomic DNA extracted as shown in Example 2, were used for the amplification of the genes of purified strains.

The amplifications were generally carried out in 10 or 20 μl of volume per sample containing buffer for the Taq polymerase (Roche or New England Biolabs) with 2.5 U of enzyme per 100 μl of final mixture. 1 pmole/μl of each primer was used in the presence of a mixture of deoxy-NTP (200 μM each).

An MJ Research PTC200 thermocycler was used, performing 30-35 cycles consisting of a denaturation at 94° C. for 30″, annealing at 58° C. for 30″, elongation at 72° C. for 30″; the cycles were preceded by an initial denaturation at 95° C. for 2′. At the end, 2 μl of each sample were analyzed on a 2% agarose gel in TAE.

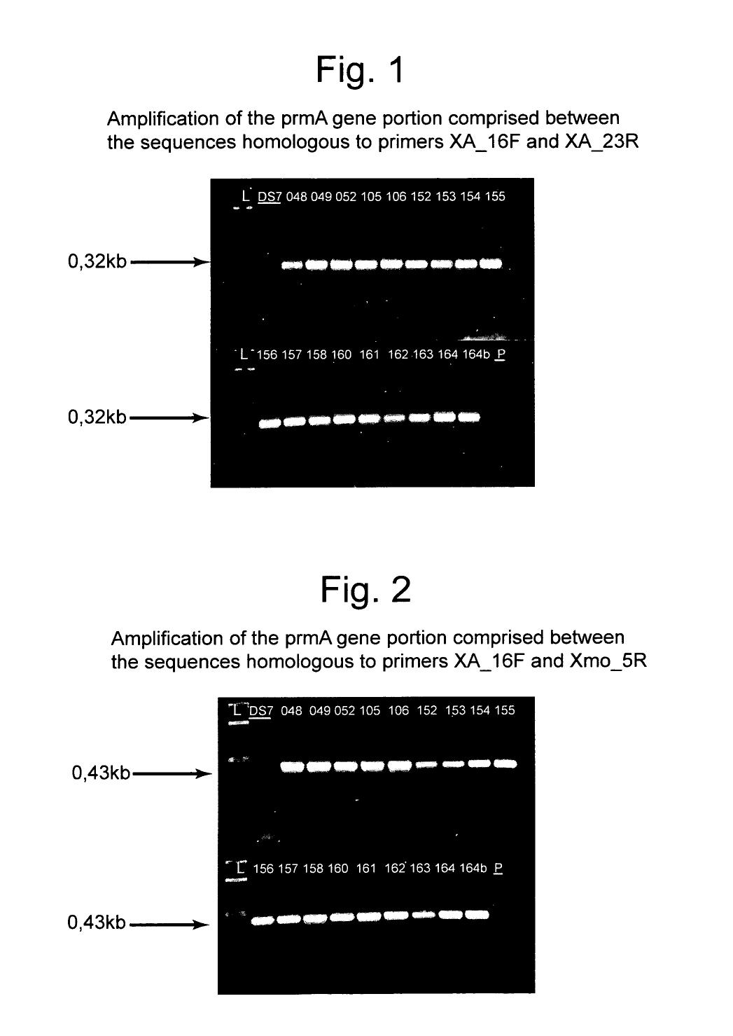

FIG. 1 shows the result of the amplification of the portion of prmA gene included in the sequences homologous to XA_{—}16F and XA_{—}23R; DS7 (Rhodococcus sp. SMV062) is a strain unable to grow on propane as the sole carbon source (negative control); P is a strain of Pseudomonas sp., isolated from an environmental sample, able to grow on N-propanol as the sole carbon source but unable to grow on propane. The following strains are from 048 and 164b respectively:

048: Gordonia sp. SMV048

049: Rhodococcus sp. SMV049

052: Rhodococcus sp. SMV052

105: Rhodococcus sp. SMV105

106: Rhodococcus sp. SMV106

152: Rhodococcus sp. SMV 152

153: Rhodococcus sp. SMV 153

154: Rhodococcus sp. SMV 154

155: Rhodococcus sp. SMV155

156: Rhodococcus sp. SMV156

157: Rhodococcus sp. SMV157

158: Mycobacterium sp. SMV158

160: Rhodococcus sp. SMV160

161: Rhodococcus sp. SMV161

162: Rhodococcus sp. SMV162

163: Gordonia sp. SMV163

164a: Rhodococcus sp. SMV164a

164b: Rhodococcus sp. SMV164b

“L” indicates the standard containing fragments of DNA of known dimensions (DNA molecular weight marker XIV-Roche).

The DNA of Rhodococcus DS7 (SMV062) and Pseudomonas sp. are not amplified under the used experimental conditions. This is in accordance with the inability of the two strains to oxidize propane.

FIG. 2 shows the result of the amplification of the portion of the prmA gene comprised between the sequences homologous to primers XA_{—}16F and Xmo_{—}5R. The samples analyzed and the conditions are identical to those of the previous experiment: also in this case Rhodococcus SMV062 (DS7) and Pseudomonas sp. do not show any amplification.

### EXAMPLE 5

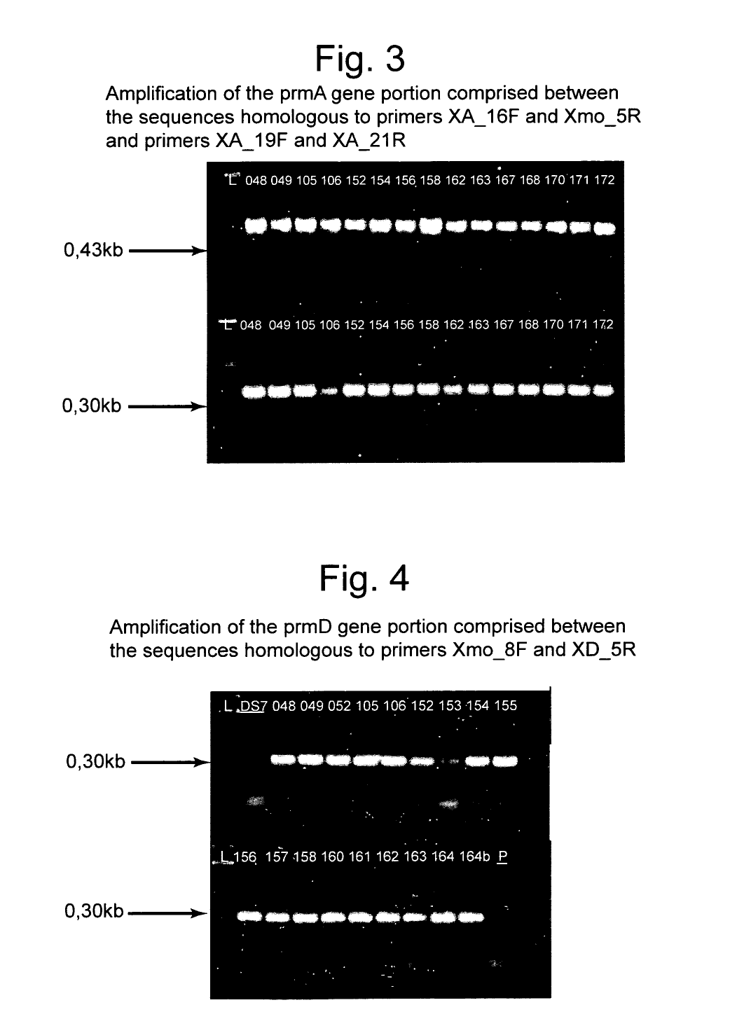

FIG. 3 shows the result of two amplification experiments of the prmA gene, carried out contemporaneously on the DNA of the strains listed hereunder:

048: Gordonia sp. SMV 048

049: Rhodococcus sp. SMV 049

105: Rhodococcus sp. SMV 105

106: Rhodococcus sp. SMV 106

152: Rhodococcus sp. SMV 152

154: Rhodococcus sp. SMV 154

156: Rhodococcus sp. SMV 156

158: Mycobacterium sp. SMV 158

162: Rhodococcus sp. SMV 162

163: Gordonia sp. SMV 163

167: Rhodococcus sp. SMV 167

168: Rhodococcus sp. SMV 168

170: Rhodococcus sp. SMV 170

171: Rhodococcus sp. SMV 171

172: Rhodococcus sp. SMV 172

The two pairs of primers used were XA_{—}16F together with Xmo_{—}5R and XA_{—}19F together with XA_{—}21R. The experimental conditions used were the same as those of the experiments described in example 4, partially modifying the cycles: after an initial denaturation at 94° C. for 2′, five cycles were carried out by incubating at the denaturation temperature of 94° C. for 30″, at the pairing temperature for 30″ and at the polymerization temperature of 72° C. for 30″; the pairing temperature was decreased by 1° C. per cycle; 31 cycles were subsequently carried out with steps of 20″ each at 94° C., 58° C. and 72° C.

| | | | | |
|---|---|---|---|---|
| 94° | C., 30″ | | 94° C., 20″ | |
| 58−>54° | C., 30″ | 5 cycles | 58° C., 20″ | 31 cycles |
| 72° | C., 30″ | | 72° C., 30″ | |

Both pairs of primers show efficiency in the amplification of the two different tracts of prmA: the different band intensity could be due to the peculiarity of each amplified sequence and to the quality of the same primers.

### EXAMPLE 6

### Amplification of the Genes prmD from Genomic DNA of Isolated Bacterial Strains

Some “universal” primers were designed from known sequences, which allow the amplification of portions of prmD genes from the purified strains listed in the sections “FORWARD PRIMER for prmD” and “REVERSE PRIMER for prmD”.

Some primers sequences which can be conveniently used for the amplification of prmD genes are the following:

Xmo_{—}8F: ACCGAGTTCTCCAACATGTG (SEQ ID NO:109)

XD_{—}5R: CCGATGTACTCGGCGGCGTC (SEQ ID NO:121)

FIG. 4 shows the result of the amplification of the portion of gene prmD comprised between the sequences homologous to primers Xmo_{—}8F and XD_{—}5R. The analyzed samples and the conditions are identical to those of the experiment of example 4: also in this case, Rhodococcus SMV062 (DS7) and Pseudomonas sp. do not show any amplification, whereas the result is positive for all the other strains.

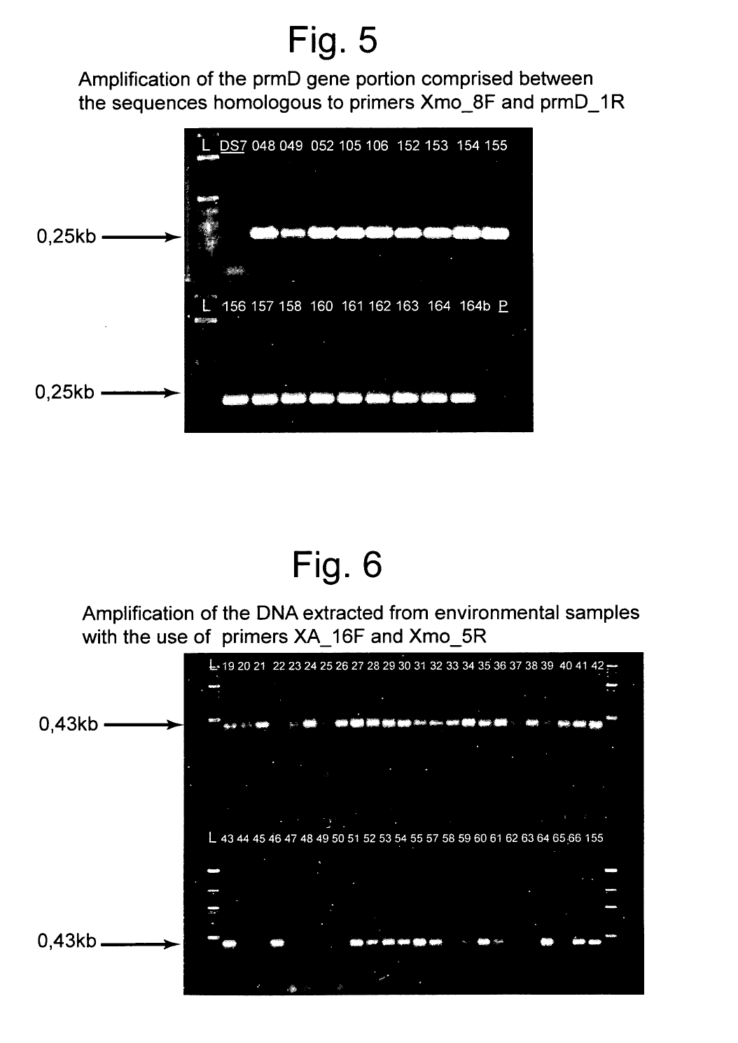

FIG. 5 shows the result of the amplification of the portion of the gene prmD comprised between the sequences homologous to primers Xmo_{—}8F and prmD_{—}1R. prmD_{—}1R is the primer described in the list “REVERSE PRIMER for prmD” with the following sequence:

| | |
|---|---|
| prmD_1R: ATGGACCATCCGNCCRTARTGNGT | (SEQ ID NO: 113) |

The analyzed samples and the conditions are identical to those of the previous experiment (relating to FIG. 4): also in this case, Rhodococcus SMV062 (DS7) and Pseudomonas sp. do not show any amplification, whereas the result is positive for all the other strains.

It can be deduced from the experiments described that specific portions of prmA and prmD genes are amplified from the DNA of all the strains isolated using propane as carbon source, as verified by the sequencing of the same amplification products. The result is similar, whether a portion of prmA or a portion of prmD is used.

### EXAMPLE 7

### Amplification of the Genes prmA from DNA Extracted from Environmental Samples with the Pair of Primers XA_{—}16F and Xmo_{—}5R

Samples of soil overlying a known oil reservoir and presumably distant samples, were analyzed using the amplification techniques of a portion of the prmA gene, previously described.

The total DNA was extracted from 0.5 g of each sample of soil, using the Q-BIOgene kit “FastDNA SPIN Kit for soil” according to the recommended protocol. At the end of the extraction, the DNA was diluted in final 200 μl of H₂O.

2 μl of a 1:10 dilution of each sample of DNA were used for the amplifications; a final 20 μl per sample of amplification contained Roche Taq polymerase buffer 1× with 2,5 U of enzyme (New England Biolabs) for each 100 μl of final mixture. 1 pmole/μl of each primer was used, in the presence of a mixture of deoxy-NTP (200 μM each).

An MJ Research PTC200 instrument was used, previously performing a denaturation at 95° C. for 2′ and 4 cycles consisting of a denaturation reaction at 94° C. for 30″, a pairing at 58° C. for 30″ with a temperature decrease of 1° C. each cycle and a polymerization at 72° C. for 30″; these were followed by 40 cycles consisting of a denaturation at 94° C. for 30″, a pairing at 58° C. for 30″ and a polymerization at 72° C. for 30″.

2.5 μl of each sample were loaded onto a 2% agarose gel in TAE.

FIG. 6 shows the photographic image of a 2% agarose gel in TAE, on which 2 μl of each sample were loaded: the order respects the number assigned during the sampling; SMV155 indicates the sample obtained from the amplification, under the same exact conditions, of about 50 pg of genomic DNA of Rhodococcus sp. SMV155.

Samples 20-32, 51-54 and 63-65 were collected in the area in which the known reservoir is comprised; samples 19, 55, 61, 62 and 64 come from areas which are approximately at the borders of the known reservoir; samples 33-43 come from an area under exploration located south with respect to the known reservoir; samples 44-50, 57-60 are all located south-east with respect to the known reservoir.

It can be said that the samples collected inside the known area of the reservoir are quite positive, giving an evident signal. The samples collected from the exploration areas also gave a variable signal, depending on the area of origin: in particular in the area located south of the known reservoir the signals are generally positive.

### EXAMPLE 8

### Amplification of the prmA Genes from DNA Extracted from Environmental Samples with the Pair of Primers XA_{—}19F/-XA_{—}21R.

An experiment analogous to that shown in example 7, was carried out on the same samples, using the primers XA_{—}19F and XA_{—}21R under identical conditions with the exception of a partial modification of the amplification cycles, according to the following scheme:

| | | | | | |
|---|---|---|---|---|---|
| 95° C., 2′ | 94° | C., 20″ | | 94° C., 20″ | |
| | 58−>54° | C., 20″ | 4 cycles | 58° C., 20″ | 28 cycles |
| | 72° | C., 20″ | | 72° C., 20″ | |

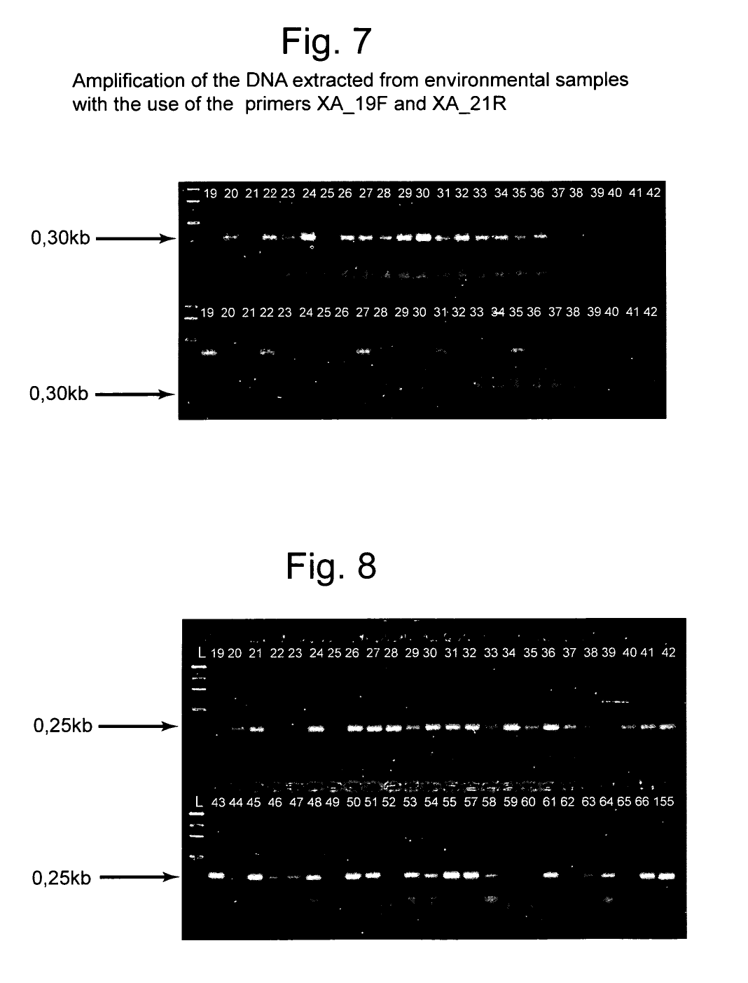

FIG. 7 shows the photograph of a 2% agarose gel on which 3 μl of each sample were loaded: the order respects the number assigned during the sampling.

Also in this case the signal is normally positive for the samples collected in the known area of the underlying reservoir.

### EXAMPLE 9

### Amplification of the prmD Genes from DNA Extracted from Environmental Samples

Analogously to the experiments of examples 7 and 8, a portion of the prmD gene was amplified, using the primers Xmo_{—}8F and prmD_{—}1R previously described.

2 μl of a 1:10 dilution of each sample of DNA were used for the amplifications; a final 10 μl per amplification sample contained Roche Taq polymerase buffer 1× with 2.5 U of enzyme (New England Biolabs) for each 100 μl of final mixture. 1 pmole/μl of each primer was used, in the presence of a mixture of Deoxy-NTP (200 μl each).

An MJ Research PTC200 instrument was used, previously performing a denaturation at 95° C. for 2′ and 10 cycles consisting of a denaturation reaction at 94° C. for 30″, a pairing at 64° C. for 30″ with a temperature decrease of 1° C. per cycle and a polymerization at 72° C. for 30″; these were followed by 40 cycles consisting of a denaturation at 94° C. for 30″, a pairing at 58° C. for 30″ and a polymerization at 72° C. for 30″.

2.5 μl of each sample were loaded on a 2% agarose gel in TAE.

FIG. 8 shows the photograph of a 2.5% agarose gel on which 3 μl of each sample were loaded: the order respects the number assigned during the sampling; SMV155 indicates the sample obtained from the amplification, under the same exact conditions, of about 50 pg of genomic DNA of Rhodococcus sp. SMV155. The result can be sufficiently superimposable with that obtained in the experiments with the pairs of primers XA_{—}16F-Xmo_{—}5R and XA-19F-XA_{—}21R; the differences may depend both on the slightly different protocol and on a different specificity of the primers themselves. The use of different pairs allows to locate the presence of propane-oxidizing bacteria in environmental samples, with a higher probability of success; in particular, the sequence of the prmA gene, showing some highly homologous regions in the different strains, is particularly suitable for the use of many pairs of primers useful for the amplification of different regions of the gene, by means of a variety of applications such DGGE and quantitative PCR (qPCR).

TABLE 1

| Primers | Sequence |
|---|---|
| Rho_1F | GGGTAGCCGGCCTGAGAG (SEQ ID NO: 126) |
| 16S_1aF | GGCAGCAGTGGGGAATATT (SEQ ID NO: 127) |
| Rho_5R | TACTCAAGTCTGCCCGTATC (SEQ ID NO: 128) |
| Rho_2F | AACAGGATTAGATACCCTGGT (SEQ ID NO: 129) |
| Rho_3R | TCGAATTAATCCACATGCTCC (SEQ ID NO: 130) |
| Rho_10F | GAGACTGCCGGGGTCAACT (SEQ ID NO: 131) |
| 16S_2R | GTCATCCCCACCTTCCTCC (SEQ ID NO: 132) |
| Rho_4R | GTGACGGGCGGTGTGTACAA (SEQ ID NO: 133) |
| >Rho_9R | CTCGCTTTCGCTACGGCTAC (SEQ ID NO: 134) |

TABLE 2

| | | Access | | |
|---|---|---|---|---|
| Strain | Protein | nr. | I° sequence | II° sequence |
| Brachymonas | butane monooxy- | AAR98534 | EWFEANYPGW | DGKTLMAQPHL |
| petroleovorans | genase: alpha sub- | | (SEQ NO: 135) | (SEQ NO: 144) |
| | unit | | | |
| Bradyrhizobium | hypothetical component | NP_770317 | EWFEHKYPGW | DGKTLVAQPHL |
| japonicum USDA | of a monooxy- | | (SEQ NO: 136) | (SEQ NO: 145) |
| 110 | genase | | | |
| Gordonia rubri- | epoxydase; alpha | BAA07114 | EWFENHYPGW | DGKTLIGOPLL |
| pertincta | subunit | | (SEQ NO: 137) | (SEQ NO: 146) |
| Gordonia sp. TY-5 | propane mono- | BAD03956 | EWFEEKYPGW | DGKTLIPQPHL |
| | oxygenase: alpha | | (SEQ NO: 138) | (SEQ NO: 147) |
| | subunit | | | |
| Mycobacterium | hypothetical alkene | AAO48576 | EWFENHYPGW | DGKTLIGOPHL |
| rhodesiae | monooxygenase: alpha | | (SEQ NO: 139) | (SEQ NO: 148) |
| | subunit | | | |
| Nocardioides sp. | probable alkene | AAV52084 | EWFENHYPGW | DGKTLMGOPHL |
| JS614 | monooxygenase: alpha | | (SEQ NO: 140) | (SEQ NO: 149) |
| | subunit | | | |
| Psaudomonas butanovora | butane monooxy- | AAM19727 | EWFEANYPGW | DGKTLIAQPHL |
| | genase hydroxy- | | (SEQ NO: 141) | (SEQ NO: 150) |
| | lasis: alpha sub- | | | |
| | unit | | | |
| Pseudonocardia | tetrahydrofuran | CAC10506 | DWFESKYPGW | DGKTLTGQPHV |
| sp. K1 | monooxygenase: alpha | | (SEQ NO: 142) | (SEQ NO: 151) |
| | sununit | | | |
| Rhodobacter | hypothetical | YP_352924 | EWFEQKYPGW | DGKTLTPQPHL |
| sphaeroides | monooxygenasis: alpha | | (SEQ NO: 143) | (SEQ NO: 152) |
| 2.4.1 | subunit | | | |

TABLE 3

| | | | |
|---|---|---|---|
| Strain | Protein | Access nr. | I° sequence |

| | | | |
|---|---|---|---|
| Acidiphilium | Hypothetical preserved | EAR39350 | STHYGRMV |
| cryptum JF-5 | protein | | (SEQ NO: 153) |
| Bradyrhizobium | Hypothetical protein | BAC48945 | STHYGRMV |
| japonicum USDA | bir 3680 | | |
| 110 | | | |
| Bradirhizobium | Hypothetical preserved | EAP31765 | STHYGRMV |
| sp. BTAi1 | protein | | |
| Frankia sp. Ccl3 | component of a | YP_481613 | STHYGRMV |
| | monooxygenase | | |
| | MmoB/DmpM | | |
| Gordonia TY-5 | propane monooxy- | BAD03959 | STHYGRMV |
| | genase; coupling | | |
| | protein | | |
| Rhodobacter | hypothetical regulating | ABA79020 | STHYGRMV |
| sphaeroides | protein | | |
| 2.4.1 | | | |
| Methylibium | coupling protein of | YP_001020150 | STHYGRMV |
| petroleiphilum | a monooxygenase | | |
| PM1 | | | |

TABLE 4

>048_prmA

(SEQ ID NO: 154)

GAGCTTGACGAAAGCCCATGCGAAGATCACCGAGTTGTCCTGGGAGCCCACCTTCGCGA
CCCCGGCCACTCGATTCGGAACCGACTACACCTTCGAGAAGGCCCCCAAGAAGGACCCG
CTGAAGCAGATCATGCGGTCGTACTTCCCGATGGAGGAGGAGAAGGACAACCGCGTGTA
CGGCGCCATGGACGGCGCCATACGCGGCAACATGTTCCGCCAGGTCCAGGAACGGTGGC
TGGAGTGGCAGAAGCTGTTCCTGTCGATCATCCCGTTCCCCGAGATCTCGGCGGCCCGC
GCGATGCCGATGGCCATCGACGCGGTGCCCAACCCGGAGATCCACAACGGGCTGGCCGT
GCAGATGATCGACGAGGTCCGTCATTCGACGATCCAGATGAACCTCAAGAAGCTGTACA
TGAACAACTACATCGACCCGGCCGGCTTCGACATCACCGAGAAGGCGTTCGCCAACAAC
TACGCCGGCACCATCGGCCGACAGTTCGGCGAGGGGTTCATCACCGGTGACGCCATCAC
GGCGGCCAACATCTACCTGACCGTCGTCGCCGAAACGGCCTTCACCAACACGCTGTTCG
TCGCGATGCCCGACGAAGCCGCCGCCAACGGCGACTACCTGCTCCCCACCGTCTTCCAC
TCGGTGCAGTCCGACGAGTCGCGGCACATCTCGAACGGCTACTCGATTCTGCTGATGGC
GCTCGCCGACGAGCGCAATCGTCCTCTGCTGGAACGTGATCTGCGGTACGCGTGGTGGA
ACAACCACTGCGTCGTCGACGCCGCGATCGGCACCTTCATCGAGTACGGCACCAAGGAC
CGGCGCAAGGACCGGGAAAGCTACGCGGAGATGTGGCGCCGGTGGATCTACGACGACTA
TTACCGCAGTTACCTTCTGCCGCTGGAGAAGTACGGGCTCACCATCCCGCACGATCTGG
TCGAGGAAGCCTGGAACCGGATCACCAACAAGCACTACGTCCACGAGGTGGCACGCTTC
TTCGCCACCGGCTGGCCGGTCAACTACTGGCGCATCGACGCCATGACCGACAAGGACTT
CGAGTGGTTCGAGGAGAAGTACCCCGGTTGGTACAACAAGTTCGGCAAGTGGTGGGAGA
ACTACAACCGCCTCGCCTACCCGGGCCGCAACAAGCCGATCGCCTTCGAGGACGTCGAT
TACGAGTACCCGCACCGCTGCTGGACCTGTATGGTGCCGTGCCTCGTCCGTGAGGACAT
GGTCGTGGACAAGGTCGACGATCAGTGGCGCACCTACTGCTCGGAGACCTGTCACTGGA
CCGACGCGGTCGCCTTCCGCGACCACTACGACGGCCGGGACACCCCGAACATGGGAAGG
CTCACCGGGTTCCGCGAATGGGAGACCCTGCATCACGGCAAGGACCTCGCCGACATCAT
CGAGGATCTGGGTTACGTCCGCGACGACGGCAAGACCCTCATCCCGCAGCCGCATCTGA
ATCTGGACCCGAAGAAGATGTGGACGCTCGACGACGTCCGCGGCAACGTCTTCAACAGT
CCCAACGTGCTGCTCAACGAGATGTCCGACGCCGAGCGGGACGCGCACATCGCGGCTTA
TCGCGCCAATCCCAACGGGGCCGTGCCGGCC
>049_prmA

(SEQ ID NO: 155)

GAGCCTGACCAAGGCCCATGCGAAGATCACCGAGCTGTCGTGGGAACCGACGTTCGCGA
CGCCGGCCACCCGCTTCGGCACCGACTACACGTTCGAGAAGGCCCCCAAGAAGGACCCT
CTCAAGCAGATCATGCGGTCCTACTTCCCCATGGAGGAGGAGAAGGACAACCGGGTCTA
CGGCGCGATGGACGGCGCCATCCGCGGGAACATGTTCCGGCAGGTCCAGCAGCGCTGGC
TGGAGTGGCAGAAGCTGTTCCTCTCGATCATCCCGTTCCCGGAGATCTCGGCGGCCCGC
GCGATGCCGATGGCCATCGACGCGGTGCCCAACCCCGAGATCCACAACGGGCTCGCCGT
GCAGATGATCGACGAGGTTCGTCACTCGACGATCCAGATGAACCTCAAGAAGCTCTACA
TGAACAACTACATCGACCCCGCCGGGTTCGACATGACCGAGAAGGCGTTCGCGAACAAC
TACGCGGGCACCATCGGCCGGCAGTTCGGGGAGGGCTTCATCACCGGTGACGCGATCAC
CGCGGCCAACATCTACCTGACCGTGGTCGCGGAGACGGCCTTCACGAACACCCTGTTCG
TGGCGATGCCCGACGAGGCGGCCGCCAACGGCGACTACCTGCTGCCCACCGTGTTCCAT
TCGGTGCAGTCCGACGAGTCGCGGCACATCTCCAACGGCTACTCGATCCTGCTCATGGC
GCTGGCCGACGAGCGGAACCGGCCGCTGCTCGAGCGGGACCTGCGCTACGCGTGGTGGA
ACAACCACTGCGTGGTGGACGCCGCGATCGGCACCTTCATCGAGTACGGCACCAAGGAC
CGCCGCAAGGATCGCGAGAGCTACGCCGAGATGTGGCGGCGGTGGATCTACGACGACTA
CTACCGCAGCTACCTCATCCCGCTCGAGAAGTACGGGCTGACCATTCCGCACGACCTCG
TCGAGGAGGCGTGGAAGCGCATCACCGAGAAGGGCTACGTCCACGAGGTGGCCCGGTTC
TTCGCGACGGGCTGGCCGGTGAACTACTGGCGGATCGACGCCATGACCGACGCGGACTT
CGAGTGGTTCGAGCACAAGTACCCGGGCTGGTATTCCAAGTACGGCAAGTGGTGGGAGA
ACTACAACCGCCTCGCCTACCCCGGCCGCAACAAGCCGATCGCGTTCGAGGAGGTGGGA
TACCAGTACCCGCACCGCTGCTGGACGTGCATGGTGCCCGCCCTCATCCGCGAGGACAT
GGTCGTGGAGAAGGTGGACGACCAGTGGCGGACCTACTGCTCGGAGACCTGCTACTGGA
CCGACGCCGTCGCGTTCCGCAGCGAGTACGAGGGACGTCCCACCCCGAACATGGGCCGC
CTCACCGGTTTCCGTGAATGGGAGACCCTGCACCACGACAAGGATCTCGCCGACATCGT
GCAGGACCTCGGGTACGTGCGCGACGACGGCAAGACCCTCGTCGGTCAGCCGCACCTCG
ACCTCGACCCGAAGAAGATGTGGACCCTCGACGACGTGCGGGGCAACACCTTCCAGAGC
CCGAACGTGTTGCTGAACCAGATGTCCGACGCAGAACGCGACGCCCACATCGCCGCGTA
CCGCGCCGGCGGCGCAGTTCCTGCC
>052_prmA

(SEQ ID NO: 156)

GAGCCTGACCAAGGCCCATGCGAAGATCACCGAGCTGTCGTGGGAACCGACGTTCGCGA
CGCCGGCCACCCGCTTCGGCACCGACTACACGTTCGAGAAGGCCCCCAAGAAGGACCCT
CTCAAGCAGATCATGCGGTCCTACTTCCCCATGGAGGAGGAGAAGGACAACCGGGTCTA
CGGCGCGATGGACGGCGCCATCCGCGGGAACATGTTCCGGCAGGTCCAGCAGCGCTGGC
TGGAGTGGCAGAAGCTGTTCCTCTCGATCATCCCGTTCCCGGAGATCTCGGCGGCCCGC
GCGATGCCGATGGCCATCGACGCGGTGCCCAACCCCGAGATCCACAACGGGCTCGCCGT
GCAGATGATCGACGAGGTTCGTCACTCGACGATCCAGATGAACCTCAAGAAGCTCTACA
TGAACAACTACATCGACCCCGCCGGGTTCGACATGACCGAGAAGGCGTTCGCGAACAAC
TACGCGGGCACCATCGGCCGGCAGTTCGGGGAGGGCTTCATCACCGGTGACGCGATCAC
CGCGGCCAACATCTACCTGACCGTGGTCGCGGAGACGGCCTTCACGAACACCCTGTTCG
TGGCGATGCCCGACGAGGCGGCCGCCAACGGCGACTACCTGCTGCCCACCGTGTTCCAT
TCGGTGCAGTCCGACGAGTCGCGGCACATCTCCAACGGCTACTCGATCCTGCTCATGGC
GCTGGCCGACGAGCGGAACCGGCCGCTGCTCGAGCGGGACCTGCGCTACGCGTGGTGGA
ACAACCACTGCGTGGTCGACGCCGCGATCGGCACCTTCATCGAGTACGGCACCAAGGAC
CGCCGCAAGGATCGCGAGAGCTATGCCGAGATGTGGCGGCGGTGGATCTACGACGACTA
CTACCGCAGCTACCTCATCCCGCTCGAGAAGTACGGGCTGACCATTCCGCACGACCTCG
TCGAGGAGGCGTGGAAGCGCATCACCGAGAAGGGCTACGTCCACGAGGTGGCCCGGTTC
TTCGCGACGGGCTGGCCGGTGAACTACTGGCGGATCGACGCCATGACCGACGCGGACTT
CGAGTGGTTCGAGCACAAGTACCCGGGCTGGTATTCCAAGTACGGCAAGTGGTGGGAGA
ACTACAACCGCCTCGCCTACCCCGGCCGCAACAAGCCGATCGCGTTCGAGGAGGTGGGA
TACCAGTACCCGCACCGCTGCTGGACGTGCATGGTGCCCGCCCTCATCCGCGAGGACAT
GGTCGTGGAGAAGGTGGACGACCAGTGGCGGACCTACTGCTCGGAGACCTGCTACTGGA
CCGACGCCGTCGCGTTCCGCAGCGAGTACGAGGGACGTCCCACCCCGAACATGGGCCGC
CTCACCGGTTTCCGTGAATGGGAGACCCTGCACCACGACAAGGATCTCGCCGACATCGT
GCAGGACCTCGGGTACGTGCGCGACGACGGCAAGACCCTCGTCGGTCAGCCGCACCTCG
ACCTCGACCCGAAGAAGATGTGGACCCTCGACGACGTGCGGGGCAACACCTTCCAGAGC
CCGAACGTGTTGCTGAACCAGATGTCCGACGCAGAGCGCGACGCCCACATCGCCGCGTA
CCGCGCCGGCGGCGCTGTTCCTGCC
>105_prmA

(SEQ ID NO: 157)

GAGCCTGACCAAGGCCCATGCGAAGATCACCGAGCTGTCGTGGGAACCGACGTTCGCGA
CGCCGGCCACCCGCTTCGGCACCGACTACACGTTCGAGAAGGCCCCCAAGAAGGACCCT
CTCAAGCAGATCATGCGGTCCTACTTCCCCATGGAGGAGGAGAAGGACAACCGGGTCTA
CGGCGCGATGGACGGCGCCATCCGCGGGAACATGTTCCGGCAGGTCCAGCAGCGCTGGC
TGGAGTGGCAGAAGCTGTTCCTCTCGATCATCCCGTTCCCGGAGATCTCGGCGGCCCGC
GCGATGCCGATGGCCATCGACGCGGTGCCCAACCCCGAGATCCACAACGGGCTCGCCGT
GCAGATGATCGACGAGGTTCGTCACTCGACGATCCAGATGAACCTCAAGAAGCTCTACA
TGAACAACTACATCGACCCCGCCGGGTTCGACATGACCGAGAAGGCGTTCGCGAACAAC
TACGCGGGCACCATCGGCCGGCAGTTCGGGGAGGGCTTCATCACCGGTGACGCGATCAC
CGCGGCCAACATCTACCTGACCGTGGTCGCGGAGACGGCCTTCACGAACACCCTGTTCG
TGGCGATGCCCGACGAGGCGGCCGCCAACGGCGACTACCTGCTGCCCACCGTGTTCCAT
TCGGTGCAGTCCGACGAGTCGCGGCACATCTCCAACGGCTACTCGATCCTGCTCATGGC
GCTGGCCGACGAGCGGAACCGGCCGCTGCTCGAGCGGGACCTGCGCTACGCGTGGTGGA
ACAACCACTGCGTGGTCGACGCCGCGATCGGCACCTTCATCGAGTACGGCACCAAGGAC
CGCCGCAAGGATCGCGAGAGCTACGCCGAGATGTGGCGGCGGTGGATCTACGACGACTA
CTACCGCAGCTACCTCATCCCGCTCGAGAAGTACGGGCTGACCATTCCGCACGACCTTG
TCGAGGAGGCGTGGAAGCGCATCACCGAGAAGGGCTACGTCCACGAGGTGGCCAGGTTC
TTCGCGACGGGCTGGCCGGTGAACTACTGGCGGATCGACGCCATGACCGACGCGGACTT
CGAGTGGTTCGAGCACAAGTACCCGGGCTGGTATTCCAAGTACGGCAAGTGGTGGGAGA
ACTACAACCGCCTCGCCTACCCCGGCCGCAACAAGCCGATCGCGTTCGAGGAGGTGGGA
TACCAGTACCCGCACCGCTGCTGGACGTGCATGGTGCCCGCCCTCATCCGCGAGGACAT
GGTCGTGGAGAAGGTGGACGACCAGTGGCGGACCTACTGCTCGGAGACCTGCTACTGGA
CCGACGCCGTCGCGTTCCGCAGCGAGTACGAGGGACGTCCCACCCCGAACATGGGCCGC
CTCACCGGTTTCCGTGAATGGGAGACCCTGCACCACGACAAGGATCTCGCCGACATCGT
GCAGGACCTCGGGTACGTGCGCGACGACGGCAAGACCCTCGTCGGTCAGCCGCATCTCG
ACCTCGACCCGAAGAAGATGTGGACCCTCGACGACGTGCGGGGCAACACCTTCCAGAGC
CCGAACGTGTTGCTGAACCAGATGTCCGACGCAGAACGCGACGCCCACATCGCCGCGTA
CCGCGCCGGCGGCGCTGTTCCTGCC
>106_prmA

(SEQ ID NO: 158)

GAGCCTGACCAAGGCCCATGCAAAGATCACCGAGCTGACGTGGGAACCGACGTTCGCGA
CGCCGGCCACCCGCTTCGGCACCGACTACACGTTCGAGAAGGCCCCCAAGAAGGACCCT
CTCAAGCAGATCATGCGGTCCTACTTCCCCATGGAGGAGGAGAAGGACAACCGGGTCTA
CGGCGCGATGGACGGCGCGATCCGCGGCAACATGTTCCGCCAGGTCCAGCAGCGCTGGC
TGGAGTGGCAGAAGCTGTTCCTCTCGATCATCCCGTTCCCGGAGATCTCCGCGGCCCGC
GCGATGCCGATGGCCATCGACGCGGTGCCCAACCCCGAGATCCACAACGGTCTCGCGGT
GCAGATGATCGACGAGGTCCGGCACTCGACGATCCAGATGAACCTCAAGAAGCTCTACA
TGAACAACTACATCGATCCCGCCGGGTTCGACATGACCGAGAAGGCGTTCGCGAACAAC
TACGCGGGCACCATCGGCCGGCAGTTCGGGGAGGGCTTCATCACCGGTGACGCGATCAC
CGCGGCCAACATCTACCTGACCGTGGTCGCGGAGACCGCCTTCACGAACACCCTGTTCG
TGGCGATGCCCGACGAGGCCGCCGCCAACGGTGACTACCTGCTGCCCACCGTGTTCCAC
TCGGTGCAGTCCGACGAGTCGCGCCACATCTCCAACGGCTACTCGATCCTGCTCATGGC
CCTGGCCGACGAGCGGAACCGGCCGCTGCTCGAACGCGACCTGCGCTACGCCTGGTGGA
ACAACCACTGCGTGGTCGACGCCGCGATCGGCACGTTCATCGAATACGGCACCAAGGAC
CGCCGCAAGGACCGCGAGAGCTACGCCGAGATGTGGCGGCGGTGGATCTACGACGACTA
CTACCGCAGCTACCTGCTCCCCCTCGAGAAGTACGGGCTCACCATTCCGCACGATCTCG
TCGAGGAGGCGTGGAAGCGCATCACCGAGAAGGGTTACGTCCACGAGGTGGCCCGGTTC
TTCGCCACGGGCTGGCCGGTGAACTACTGGCGGATCGACGCCATGACCGACGCGGACTT
CGAGTGGTTCGAGGAGAAGTACCCCGGCTGGTACTCCAAGTTCGGCAAGTGGTGGGAGA
ACTACAACCGCCTCGCCTACCCCGGCCGCAACAAGCCGATCGCGTTCGAGGAAGTCGGA
TACCAGTACCCGCACCGCTGCTGGACCTGCATGGTGCCGGCCCTGGTCCGCGAGGACAT
GGTCGTGGAGAAGGTCGACGACCAGTGGCGGACCTACTGCTCGGAGACGTGCTACTGGA
CCGACGCGGTCGCCTTCCGCGGTGAGTACGAGGGCCGGCCCACGCCGAACATGGGCCGT
CTCACCGGTTTCCGGGAATGGGAGACCCTGCACCACGACAAGGATCTCGCCGACATCGT
GCAGGACCTCGGGTATGTGCGCGACGACGGCAAGACCCTCGTCGGCCAGCCGCACCTCG
ATCTCGACCCGAAGAAGATGTGGACCCTCGACGACGTGCGGGGCAACACCTTCCAGAGC
CCGAACGTCCTGCTGAACCAGATGACGGACGAGGAGCGCGCAGCGCACATCGCGGAGTA
CCGCGCCGGCGCCACGCCGCTC
>152_prmA

(SEQ ID NO: 159)

AAGCCTGACAAAGGCCCACGCGAAGATCACCGAACTGTCATGGGATCCGACATTCGCAA
CCCCGGCCACCCGGTTCGGCACCGACTACACCTTCGAGAAGGCTCCGAAGAAGGATCCT
CTCAAGCAGATCATGCGGTCCTACTTCCCGATGGAGGAAGAGAAGGACAACCGCGTGTA
CGGCGCCATGGACGGTGCCATCCGCGGCAACATGTTCCGCCAGGTGCAGCAGCGGTGGC
TCGAATGGCAGAAGCTGTTCCTGTCGATCATTCCGTTCCCGGAGATCTCGGCCGCCCGA
GCGATGCCGATGGCCATCGACGCCGTCCCCAACCCGGAGATCCACAACGGGCTGGCGGT
GCAGATGATCGACGAGGTTCGTCACTCGACGATCCAGATGAACCTCAAGAAGCTGTACA
TGAACAACTACATCGATCCCGCCGGTTTCGACATGACCGAGAAGGCGTTCGCGAACAAC
TACGCGGGGACCATCGGCAGGCAGTTCGGTGAAGGGTTCATCACCGGCGACGCGATCAC
CGCGGCGAACATCTATCTGACCGTGGTCGCCGAGACGGCGTTCACCAACACCCTGTTCG
TTGCCATGCCCGACGAGGCGGCCGCCAACGGTGACTACCTGTTGCCGACGGTCTTCCAC
TCGGTGCAGTCGGACGAGTCGCGGCACATCTCCAACGGCTATTCGATCCTGCTGATGGC
ACTCGCCGACGAGCGCAACCGTCCACTGCTCGAACGTGACCTGCGGTACGCGTGGTGGA
ACAACCACTGCGTCGTCGACGCCGCGATCGGCACCTTCATCGAGTACGGCACGAAGGAC
CGCCGCAAGGACCGGGAGAGTTACGCCGAGATGTGGCGGCGGTGGATCTACGACGATTA
CTACCGCAGCTACCTCATCCCGCTCGAGAAGTACGGCCTGACGATCCCGCACGACCTGG
TCGAGGAGGCGTGGAAGCGGATCACCGACAAGGGCTACGTCCACGAGGTGGCCCGGTTC
TTCGCCACCGGATGGCCGGTGAACTACTGGCGGATCGACGCGATGACCGACAAGGACTT
CGAGTGGTTCGAGCACAAGTACCCGGGCTGGTACTCGAAGTACGGCAAGTGGTGGGAGG
AGTACAACCGGCTCGCCTACCCCGGTCGCAACAAGCCGATCGCGTTCGAGGAGGTCGGA
TACCAGTACCCGCACCGGTGCTGGACCTGCATGGTTCCCGCCCTCATCCGTGAGGACAT
GGTCGTGGAGAAGGTCGACGAGCAGTGGCGGACCTATTGCTCGGAAACCTGCTACTGGA
CCGACGCCGTCGCGTTCCGCAGCGAGTACCAGGGCCGCCCGACCCCGAACATGGGCCGG
CTCACGGGATTCCGGGAGTGGGAAACCCTGCATCACGGCAAGGACCTCGCCGACATCGT
CTCCGATCTCGGCTACGTCCGCGACGACGGCAAGACCCTGGTCGGTCAGCCGCACCTCG
ATCTGGACGATCCGAAGAAGATGTGGACTCTCGACGACGTGCGGGGCAACACCTTCCAG
AGCCCGAACGTGCTCTTGAACGAGATGTCCGACGCCGACCGCAACGCGCACATCGCCGC
GTACCGCGCCGGCGGCCCAGTTCCGGCC
>154_prmA

(SEQ ID NO: 160)

AAGCCTGACAAAGGCCCACGCGAAGATCACCGAACTGTCATGGGATCCGACATTCGCAA
CCCCGGCCACCCGGTTCGGCACCGACTACACCTTCGAGAAGGCTCCGAAGAAGGATCCT
CTCAAGCAGATCATGCGGTCCTACTTCCCGATGGAGGAAGAGAAGGACAACCGCGTGTA
CGGCGCCATGGACGGTGCCATCCGCGGCAACATGTTCCGCCAGGTGCAGCAGCGGTGGC
TCGAATGGCAGAAGCTGTTCCTGTCGATCATTCCGTTCCCGGAGATCTCGGCCGCCCGA
GCGATGCCGATGGCCATCGACGCCGTCCCCAACCCGGAGATCCACAACGGGCTGGCGGT
GCAGATGATCGACGAGGTTCGTCACTCGACGATCCAGATGAACCTCAAGAAGCTGTACA
TGAACAACTACATCGATCCCGCCGGTTTCGACATGACCGAGAAGGCGTTCGCGAACAAC
TACGCGGGGACCATCGGCAGGCAGTTCGGTGAAGGGTTCATCACCGGCGACGCGATCAC
CGCGGCGAACATCTATCTGACCGTGGTCGCCGAGACGGCGTTCACCAACACCCTGTTCG
TTGCCATGCCCGACGAGGCGGCCGCCAACGGTGACTACCTGTTGCCGACGGTCTTCCAC
TCGGTGCAGTCGGACGAGTCGCGGCACATCTCCAACGGCTATTCGATCCTGCTGATGGC
ACTCGCCGACGAGCGCAACCGTCCACTACTCGAACGTGACCTGCGGTACGCGTGGTGGA
ACAACCACTGCGTCGTCGACGCCGCGATCGGCACCTTCATCGAGTACGGCACGAAGGAC
CGCCGCAAGGACCGGGAGAGTTACGCCGAGATGTGGCGGCGGTGGATCTACGACGATTA
CTACCGCAGCTACCTCATCCCGCTCGAGAAGTACGGCCTGACGATCCCGCACGACCTGG
TCGAGGAGGCGTGGAAGCGGATCACCGACAAGGGCTACGTCCACGAGGTGGCCCGGTTC
TTCGCCACCGGATGGCCGGTGAACTACTGGCGGATCGACGCGATGACCGACAAGGACTT
CGAGTGGTTCGAGCACAAGTACCCGGGCTGGTACTCGAAGTACGGCAAGTGGTGGGAGG
AGTACAACCGGCTCGCCTACCCCGGTCGCAACAAGCCGATCGCGTTCGAGGAGGTCGGA
TACCAGTACCCGCACCGGTGCTGGACCTGCATGGTTCCCGCCCTCATCCGTGAGGACAT
GGTCGTGGAGAAGGTCGACGAGCAGTGGCGGACCTACTGCTCGGAAACCTGCTACTGGA
CCGACGCCGTCGCGTTCCGCAGCGAGTACCAGGGCCGCCCGACCCCGAACATGGGCCGG
CTCACGGGATTCCGGGAGTGGGAAACCCTGCATCACGGCAAGGACCTCGCCGACATCGT
CTCCGATCTCGGCTACGTCCGCGACGACGGCAAGACCCTGGTCGGTCAGCCGCACCTCG
ATCTGGACGATCCGAAGAAGATGTGGACTCTCGACGACGTGCGGGGCAACACCTTCCAG
AGCCCGAACGTGCTCTTGAACGAGATGTCCGACGCCGACCGCAACGCGCACATCGCCGC
GTACCGCGCCGGCGGCGCAGTTCCGGCC
>155_prmA

(SEQ ID NO: 161)

AAGCCTGACAAAGGCCCACGCGAAGATCACCGAACTGTCATGGGATCCGACATTCGCAA
CCCCGGCCACCCGGTTCGGCACCGACTACACCTTCGAGAAGGCTCCGAAGAAGGATCCT
CTCAAGCAGATCATGCGGTCCTACTTCCCGATGGAGGAAGAGAAGGACAACCGCGTGTA
CGGCGCCATGGACGGTGCCATCCGCGGCAACATGTTCCGCCAGGTGCAGCAGCGGTGGC
TCGAATGGCAGAAGCTGTTCCTGTCGATCATTCCGTTCCCGGAGATCTCGGCCGCCCGA
GCGATGCCGATGGCCATCGACGCCGTCCCCAACCCGGAGATCCACAACGGGCTGGCGGT
GCAGATGATCGACGAGGTTCGTCACTCGACGATCCAGATGAACCTCAAGAAGCTGTACA
TGAACAACTACATCGATCCCGCCGGTTTCGACATGACCGAGAAGGCGTTCGCGAACAAC
TACGCGGGGACCATCGGCAGGCAGTTCGGTGAAGGGTTCATCACCGGCGACGCGATCAC
CGCGGCGAACATCTATCTGACCGTGGTCGCCGAGACGGCGTTCACCAACACCCTGTTCG
TTGCCATGCCCGACGAGGCGGCCGCCAACGGTGACTACCTGTTGCCGACGGTCTTCCAC
TCGGTGCAGTCGGACGAGTCGCGGCACATCTCCAACGGCTATTCGATCCTGCTGATGGC
ACTCGCCGACGAGCGCAACCGTCCACTGCTCGAACGTGACCTGCGGTACGCGTGGTGGA
ACAACCACTGCGTCGTCGACGCCGCGATCGGCACCTTCATCGAGTACGGCACGAAGGAC
CGCCGCAAGGACCGGGAGAGTTACGCCGAGATGTGGCGGCGGTGGATCTACGACGATTA
CTACCGCAGCTACCTCATCCCGCTCGAGAAGTACGGCCTGACGATCCCGCACGACCTGG
TCGAGGAGGCGTGGAAGCGGATCACCGACAAGGGCTACGTCCACGAGGTGGCCCGGTTC
TTCGCCACCGGATGGCCGGTGAACTACTGGCGGATCGACGCGATGACCGACAAGGACTT
CGAGTGGTTCGAGCACAAGTACCCGGGCTGGTACTCGAAGTACGGCAAGTGGTGGGAGG
AGTACAACCGGCTCGCCTACCCCGGTCGCAACAAGCCGATCGCGTTCGAGGAGGTCGGA
TACCAGTACCCGCACCGGTGCTGGACCTGCATGGTTCCCGCCCTCATCCGTGAGGACAT
GGTCGTGGAGAAGGTCGACGAGCAGTGGCGGACCTATTGCTCGGAAACCTGCTACTGGA
CCGACGCCGTCGCGTTCCGCAGCGAGTACCAGGGCCGCCCGACCCCGAACATGGGCCGG
CTCACGGGATTCCGGGAGTGGGAAACCCTGCATCACGGCAAGGACCTCGCCGACATCGT
CTCCGATCTCGGCTACGTCCGCGACGACGGCAAGACCCTGGTCGGTCAGCCGCACCTCG
ATCTGGACGATCCGAAGAAGATGTGGACTCTCGACGACGTGCGGGGCAACACCTTCCAG
AGCCCGAACGTGCTCTTGAACGAGATGTCCGACGCCGACCGCAACGCGCACATCGCCGC
GTACCGCGCCGGCGGCCCAGTTCCGGCC
>156_prmA

(SEQ ID NO: 162)

AAGCTTGACAAAGGCCCACGCGAAGATCACCGAACTGTCATGGGATCCGACCTTCGCGA
CCCCGGCCACCCGGTTCGGCACCGACTACACCTTCGAGAAGGCTCCGAAGAAGGACCCT
CTCAAGCAGATCATGCGGTCGTACTTCCCGATGGAGGAAGAGAAGGACAACCGCGTGTA
CGGCGCCATGGACGGCGCCATCCGCGGCAACATGTTCCGCCAGGTTCAGCAGCGCTGGC
TCGAATGGCAGAAGCTGTTCCTGTCGATCATTCCGTTCCCGGAAATCTCGGCCGCCCGT
GCGATGCCGATGGCCATCGACGCCGTGCCGAACCCGGAGATTCACAACGGGCTCGCGGT
GCAGATGATCGACGAGGTTCGGCACTCGACGATCCAGATGAACCTCAAGAAGCTGTACA
TGAACAACTACATCGATCCCGCCGGGTTCGATATGACGGAGAAGGCGTTCGCGAACAAC
TACGCCGGCACCATCGGCCGTCAGTTCGGCGAAGGCTTCATTACCGGCGACGCGATCAC
CTCGGCGAACATCTACCTGACCGTGGTTGCCGAAACTGCGTTCACGAACACCCTGTTCG
TGGCCATGCCCGACGAGGCCGCCGCCAATGGTGATTACCTGCTGCCCACTGTGTTTCAC
TCGGTGCAGTCCGACGAATCACGACACATCTCCAACGGTTACTCGATCCTGTTGATGGC
CCTCGCCGACGAGCGCAACCGTCCCCTGCTCGAACGCGACTTGCGGTACGCGTGGTGGA
ACAACCATTGCGTCGTCGACGCCGCGATCGGCACCTTCATCGAGTACGGCACCAAGGAC
CGTCGCAAGGACCGGGAAAGCTACGCCGAGATGTGGCGGCGGTGGATCTACGACGATTA
CTACCGCAGCTACCTCATCCCGCTCGAAAAGTACGGCCTGACCATCCCGCACGACCTGG
TCGAAGAGGCGTGGAAGCGGATCACCGAAAAGGGTTACGTCCACGAGGTAGCGCGTTTC
TTCGCCACCGGGTGGCCGGTCAACTACTGGCGGATCGATGCGATGACCGACAAGGACTT
CGAGTGGTTCGAGCACAAGTACCCGGGCTGGTACTCGAAGTACGGCAAGTGGTGGGAGG
AGTACAACCGCCTCGCCTACCCGGGACGCAACAAGCCGATCGCGTTCGAGGAGGTCGGG
TACCAGTACCCGCACCGCTGCTGGACGTGCATGGTGCCCGCGCTCATCCGTGAGGACAT
GGTCGTGGAGAAGGTCGACGAGCAGTGGCGGACCTACTGCTCGGAGACCTGCTACTGGA
CCGACGCCGTCGCGTTCCGCAGCGAGTACCAGGGCAAGCCGACTCCGAACATGGGGCGG
CTCACCGGCTTCCGTGAATGGGAGACCCTGCATCACGGTAAGGACCTCGCTGACATCGT
GCAGGACCTGGGTTATGTCCGCGACGACGGCAAGACCCTCGTCGGTCAGCCGCACCTGC
ACCTGGACGACCCGAAGAAGTTGTGGACTCTCGACGACGTCCGCGGCAACACGTTCCAG
AGCCCGAACGTGCTCTTGAACGAGATGTCGGACGCCGAACGCAACGCGCACATTGCCGC
GTACCGCGCCGGCGGCGCAGTTCCGGCC
>156_prmA

(SEQ ID NO: 162)

AAGCTTGACAAAGGCCCACGCGAAGATCACCGAACTGTCATGGGATCCGACCTTCGCGA
CCCCGGCCACCCGGTTCGGCACCGACTACACCTTCGAGAAGGCTCCGAAGAAGGACCCT
CTCAAGCAGATCATGCGGTCGTACTTCCCGATGGAGGAAGAGAAGGACAACCGCGTGTA
CGGCGCCATGGACGGCGCCATCCGCGGCAACATGTTCCGCCAGGTTCAGCAGCGCTGGC
TCGAATGGCAGAAGCTGTTCCTGTCGATCATTCCGTTCCCGGAAATCTCGGCCGCCCGT
GCGATGCCGATGGCCATCGACGCCGTGCCGAACCCGGAGATTCACAACGGGCTCGCGGT
GCAGATGATCGACGAGGTTCGGCACTCGACGATCCAGATGAACCTCAAGAAGCTGTACA
TGAACAACTACATCGATCCCGCCGGGTTCGATATGACGGAGAAGGCGTTCGCGAACAAC
TACGCCGGCACCATCGGCCGTCAGTTCGGCGAAGGCTTCATTACCGGCGACGCGATCAC
CTCGGCGAACATCTACCTGACCGTGGTTGCCGAAACTGCGTTCACGAACACCCTGTTCG
TGGCCATGCCCGACGAGGCCGCCGCCAATGGTGATTACCTGCTGCCCACTGTGTTTCAC
TCGGTGCAGTCCGACGAATCACGACACATCTCCAACGGTTACTCGATCCTGTTGATGGC
CCTCGCCGACGAGCGCAACCGTCCCCTGCTCGAACGCGACTTGCGGTACGCGTGGTGGA
ACAACCATTGCGTCGTCGACGCCGCGATCGGCACCTTCATCGAGTACGGCACCAAGGAC
CGTCGCAAGGACCGGGAAAGCTACGCCGAGATGTGGCGGCGGTGGATCTACGACGATTA
CTACCGCAGCTACCTCATCCCGCTCGAAAAGTACGGCCTGACCATCCCGCACGACCTGG
TCGAAGAGGCGTGGAAGCGGATCACCGAAAAGGGTTACGTCCACGAGGTAGCGCGTTTC
TTCGCCACCGGGTGGCCGGTCAACTACTGGCGGATCGATGCGATGACCGACAAGGACTT
CGAGTGGTTCGAGCACAAGTACCCGGGCTGGTACTCGAAGTACGGCAAGTGGTGGGAGG
AGTACAACCGCCTCGCCTACCCGGGACGCAACAAGCCGATCGCGTTCGAGGAGGTCGGG
TACCAGTACCCGCACCGCTGCTGGACGTGCATGGTGCCCGCGCTCATCCGTGAGGACAT
GGTCGTGGAGAAGGTCGACGAGCAGTGGCGGACCTACTGCTCGGAGACCTGCTACTGGA
CCGACGCCGTCGCGTTCCGCAGCGAGTACCAGGGCAAGCCGACTCCGAACATGGGGCGG
CTCACCGGCTTCCGTGAATGGGAGACCCTGCATCACGGTAAGGACCTCGCTGACATCGT
GCAGGACCTGGGTTATGTCCGCGACGACGGCAAGACCCTCGTCGGTCAGCCGCACCTGC
ACCTGGACGACCCGAAGAAGTTGTGGACTCTCGACGACGTCCGCGGCAACACGTTCCAG
AGCCCGAACGTGCTCTTGAACGAGATGTCGGACGCCGAACGCAACGCGCACATTGCCGC
GTACCGCGCCGGCGGCGCAGTTCCGGCC
>157_prmA

(SEQ ID NO: 163)

AAGCCTGACAAAGGCCCACGCGAAGATCACCGAACTGTCATGGGATCCGACATTCGCAA
CCCCGGCCACCCGGTTCGGCACCGACTACACCTTCGAGAAGGCTCCGAAGAAGGATCCT
CTCAAGCAGATCATGCGGTCCTACTTCCCGATGGAGGAAGAGAAGGACAACCGCGTGTA
CGGCGCCATGGACGGTGCCATCCGCGGCAACATGTTCCGCCAGGTGCAGCAGCGGTGGC
TCGAATGGCAGAAGCTGTTCCTGTCGATCATTCCGTTCCCGGAGATCTCGGCCGCCCGA
GCGATGCCGATGGCCATCGACGCCGTCCCCAACCCGGAGATCCACAACGGGCTGGCGGT
GCAGATGATCGACGAGGTTCGTCACTCGACGATCCAGATGAACCTCAAGAAGCTGTACA
TGAACAACTACATCGATCCCGCCGGTTTCGACATGACCGAGAAGGCGTTCGCGAACAAC
TACGCGGGGACCATCGGCAGGCAGTTCGGTGAAGGGTTCATCACCGGCGACGCGATCAC
CGCGGCGAACATCTATCTGACCGTGGTCGCCGAGACGGCGTTCACCAACACCCTGTTCG
TTGCCATGCCCGACGAGGCGGCCGCCAACGGTGACTACCTGTTGCCGACGGTCTTCCAC
TCGGTGCAGTCGGACGAGTCGCGGCACATCTCCAACGGCTATTCGATCCTGCTGATGGC
ACTCGCCGACGAGCGCAACCGTCCACTGCTCGAACGTGACCTGCGGTACGCGTGGTGGA
ACAACCACTGCGTCGTCGACGCCGCGATCGGCACCTTCATCGAGTACGGCACGAAGGAC
CGCCGCAAGGACCGGGAGAGTTACGCCGAGATGTGGCGGCGGTGGATCTACGACGATTA
CTACCGCAGCTACCTCATCCCGCTCGAGAAGTACGGCCTGACGATCCCGCACGACCTGG
TCGAGGAGGCGTGGAAGCGGATCACCGACAAGGGCTACGTCCACGAGGTGGCCCGGTTC
TTCGCCACCGGATGGCCGGTGAACTACTGGCGGATCGACGCGATGACCGACAAGGACTT
CGAGTGGTTCGAGCACAAGTACCCGGGCTGGTACTCGAAGTACGGCAAGTGGTGGGAGG
AGTACAACCGGCTCGCCTACCCCGGTCGCAACAAGCCGATCGCGTTCGAGGAGGTCGGA
TACCAGTACCCGCACCGGTGCTGGACCTGCATGGTTCCCGCCCTCATCCGTGAGGACAT
GGTCGTGGAGAAGGTCGACGAGCAGTGGCGGACCTATTGCTCGGAAACCTGCTACTGGA
CCGACGCCGTCGCGTTCCGCAGCGAGTACCAGGGCCGCCCGACCCCGAACATGGGCCGG
CTCACGGGATTCCGGGAGTGGGAAACCCTGCATCACGGCAAGGACCTCGCCGACATCGT
CTCCGATCTCGGCTACGTCCGCGACGACGGCAAGACCCTGGTCGGTCAGCCGCACCTCG
ATCTGGACGATCCGAAGAAGATGTGGACTCTCGACGACGTGCGGGGCAACACCTTCCAG
AGCCCGAACGTGCTCTTGAACGAGATGTCCGACGCCGACCGCAACGCGCACATCGCCGC
GTACCGCGCCGGCGGCCCAGTTCCGGCC
>158_prmA

(SEQ ID NO: 164)

AAGCCTGACCAAGGCGCACGCGAAGATCACCGAGCTGTCGTGGGAACCGACGTTCGCCA
CGCCCGCCACCCGTTTCGGCACCGACTACACCTTCGAGAAGGCCCCGAAGAAGGACCCG
CTCAAGCAGATCATGCGGTCCTACTTCCCCATGGAGGAGGAGAAGGACAACCGCGTCTA
CGGCGCCATGGACGGCGCCATCCGCGGCAACATGTTCCGCCAGGTGCAGCAGCGCTGGC
TGGAGTGGCAGAAGTTGTTCCTGTCCATCATCCCGTTCCCGGAGATCTCGGCGGCGCGG
GCCATGCCCATGGCCATCGACGCCGTGCCCAATCCCGAGATCCACAACGGGCTGGCGGT
CCAGATGATCGACGAGGTCCGGCACTCGACGATCCAGATGAACCTCAAGAAGCTGTACA
TGAACAACTACATCGACCCCGCCGGTTTCGACATCACCGAGAAGGCGTTCGCCAACAAC
TACGCCGGCACCATCGGCCGCCAGTTCGGCGAGGGCTTCATCACCGGCGACGCGATCAC
CGCCGCCAACATTTATCTGACCGTGGTGGCCGAAACCGCCTTCACCAACACACTTTTCG
TGGCCATGCCGGACGAGGCCGCGGCCAACGGCGACTATCTGCTGCCGACGGTGTTCCAC
TCGGTGCAGTCCGATGAGTCCCGCCACATCTCCAACGGCTACTCGATCCTGTTGATGGC
ACTGGCCGACGAGCGCAACCGCCCCCTGCTGGAACGCGACCTGCGTTACGCCTGGTGGA
ACAACCACTGCGTGGTCGACGCGGCCATCGGCACCTTCATCGAGTACGGCACCAAGGAC
CGCCGCAAGGACCGGGAGAGCTACGCCGAGATGTGGCGGCGCTGGATCTACGACGACTA
CTACCGCAGTTACCTGCTGCCGCTGGAGAAGTACGGCCTGACCATTCCACACGACCTGG
TGGAGGAGGCGTGGAAGCGCATCGTCGACAAGCACTACGTGCACGAGGTGGCCCGCTTC
TTCGCCACCGGATGGCCGGTCAACTACTGGCGCATCGATGCCATGACCGACAAGGACTT
CGAGTGGTTCGAGGAGAAGTACCCCGGCTGGTACAACAAGTTCGGCCGCTGGTGGGAGG
ACTACAACCGGCTGGCCTACCCGGGCCGCAACAAGCCGATCGCCTTCGAAGAGGTGGGC
TATCAGTACCCGCACCGCTGCTGGACCTGCATGGTGCCGGCGCTGATCCGCGAGGACAT
GGTGGTGGAGAAGGTCGACGACCAGTGGCGCACCTACTGCTCGGAGACCTGCTACTGGA
CCGATGCGGTGGCCTTCCGCGGTGAGTACGAGGGCCGGCCGACGCCGAACATGGGCCGG
CTCACCGGTTTCCGCGAGTGGGAGACCCTGCACCACGGCAAGGACCTGGCCGACATCGT
CGCCGACCTCGGTTATGTGCGCGACGACGGCAAGACCCTGATCCCGCAGCCGCACCTGG
ATCTGGACCCCAAGAAGATGTGGACCCTCGACGACGTGCGCGGCAACGTCTTCAACAGC
CCCAACGTGCTGCTCAACGAGATGAGTGATGCCGAACGGGACGCCCACGTCGCGGCCTA
CCGCGCTGGT
>160_prmA

(SEQ ID NO: 165)

AAGCCTGACAAAGGCCCACGCGAAGATCACCGAACTGTCATGGGATCCGACATTCGCAA
CCCCGGCCACCCGGTTCGGCACCGACTACACCTTCGAGAAGGCTCCGAAGAAGGATCCT
CTCAAGCAGATCATGCGGTCCTACTTCCCGATGGAGGAAGAGAAGGACAACCGCGTGTA
CGGCGCCATGGACGGTGCCATCCGCGGCAACATGTTCCGCCAGGTGCAGCAGCGGTGGC
TCGAATGGCAGAAGCTGTTCCTGTCGATCATTCCGTTCCCGGAGATCTCGGCCGCCCGA
GCGATGCCGATGGCCATCGACGCCGTCCCCAACCCGGAGATCCACAACGGGCTGGCGGT
GCAGATGATCGACGAGGTTCGTCACTCGACGATCCAGATGAACCTCAAGAAGCTGTACA
TGAACAACTACATCGATCCCGCCGGTTTCGACATGACCGAGAAGGCGTTCGCGAACAAC
TACGCGGGGACCATCGGCAGGCAGTTCGGTGAAGGGTTCATCACCGGCGACGCGATCAC
CGCGGCGAACATCTATCTGACCGTGGTCGCCGAGACGGCGTTCACCAACACCCTGTTCG
TTGCCATGCCCGACGAGGCGGCCGCCAACGGTGACTACCTGTTGCCGACGGTCTTCCAC
TCGGTGCAGTCGGACGAGTCGCGGCACATCTCCAACGGCTATTCGATCCTGCTGATGGC
ACTCGCCGACGAGCGCAACCGTCCACTGCTCGAACGTGACCTTCGGTACGCGTGGTGGA
ACAACCACTGCGTCGTCGACGCCGCGATCGGCACCTTCATCGAGTACGGCACGAAGGAC
CGCCGCAAGGACCGGGAGAGTTACGCCGAGATGTGGCGGCGGTGGATCTACGACGATTA
CTACCGCAGCTACCTCATCCCGCTCGAGAAGTACGGCCTGACGATCCCGCACGACCTGG
TCGAGGAGGCGTGGAAGCGGATCACCGACAAGGGCTACGTCCACGAGGTGGCCCGGTTC
TTCGCCACCGGATGGCCGGTGAACTACTGGCGGATCGACGCGATGACCGACAAGGACTT
CGAGTGGTTCGAGCACAAGTACCCGGGCTGGTACTCGAAGTACGGCAAGTGGTGGGAGG
AGTACAACCGGCTCGCCTACCCCGGTCGCAACAAGCCGATCGCGTTCGAGGAGGTCGGA
TACCAGTACCCGCACCGGTGCTGGACCTGCATGGTTCCCGCACTCATCCGTGAGGACAT
GGTCGTGGAGAAGGTCGACGAGCAGTGGCGGACCTACTGCTCGGAAACCTGCTACTGGA
CCGACGCCGTCGCGTTCCGCAGCGAGTACCAGGGCCGCCCGACCCCGAACATGGGCCGG
CTCAA
>161_prmA

(SEQ ID NO: 166)

GAGCCTGACCAAGGCCCATGCGAAGATCACCGAGCTGTCGTGGGAACCGACGTTCGCGA
CGCCGGCCACCCGCTTCGGCACCGACTACACGTTCGAGAAGGCCCCCAAGAAGGACCCT
CTCAAGCAGATCATGCGGTCCTACTTCCCCATGGAGGAGGAGAAGGACAACCGGGTCTA
CGGCGCGATGGACGGCGCCATCCGCGGGAACATGTTCCGGCAGGTCCAGCAGCGCTGGC
TGGAGTGGCAGAAGCTGTTCCTCTCGATCATCCCGTTCCCGGAGATCTCGGCGGCCCGC
GCGATGCCGATGGCCATCGACGCGGTGCCCAACCCCGAGATCCACAACGGGCTCGCCGT
GCAGATGATCGACGAGGTTCGTCACTCGACGATCCAGATGAACCTCAAGAAGCTCTACA
TGAACAACTACATCGACCCCGCCGGGTTCGACATGACCGAGAAGGCGTTCGCGAACAAC
TACGCGGGCACCATCGGCCGGCAGTTCGGGGAGGGCTTCATCACCGGTGACGCGATCAC
CGCGGCCAACATCTACCTGACCGTGGTCGCGGAGACGGCCTTCACGAACACCCTGTTCG
TGGCGATGCCCGACGAGGCGGCCGCCAACGGCGACTACCTGCTGCCCACCGTGTTCCAT
TCGGTGCAGTCCGACGAGTCGCGGCACATCTCCAACGGCTACTCGATCCTGCTCATGGC
GCTGGCCGACGAGCGGAACCGGCCGCTGCTCGAGCGGGACCTGCGCTACGCGTGGTGGA
ACAACCACTGCGTGGTCGACGCCGCGATCGGCACCTTCATCGAGTACGGCACCAAGGAC
CGCCGCAAGGATCGCGAGAGCTACGCCGAGATGTGGCGGCGGTGGATCTACGACGACTA
CTACCGCAGCTACCTCATCCCGCTCGAGAAGTACGGGCTGACCATTCCGCACGACCTCG
TCGAGGAGGCGTGGAAGCGCATCACCGAGAAGGGCTACGTCCACGAGGTGGCCCGGTTC
TTCGCGACGGGCTGGCCGGTGAACTACTGGCGGATCGACGCCATGACCGACGCGGACTT
CGAGTGGTTCGAGCACAAGTACCCGGGCTGGTATTCCAAGTACGGCAAGTGGTGGGAGA
ACTACAACCGCCTCGCCTACCCCGGCCGCAACAAGCCGATAGCGTTCGAGGAGGTGGGA
TACCAGTACCCGCACCGCTGCTGGACGTGCATGGTGCCCGCCCTCATCCGCGAGGACAT
GGTCGTGGAGAAGGTGGACGACCAGTGGCGGACCTACTGCTCGGAGACCTGCTACTGGA
CCGACGCCGTCGCGTTCCGCAGCGAGTACGAGGGACGTCCCACCCCGAACATGGGCCGC
CTCACCGGTTTCCGTGAATGGGAGACCCTGCACCACGACAAGGATCTCGCCGACATCGT
GCAGGACCTCGGGTACGTGCGCGACGACGGCAAGACCCTCGTCGGTCAGCCGCATCTCG
ACCTCGACCCGAAGAAGATGTGGACCCTCGACGACGTGCGGGGCAACACCTTCCAGAGC
CCGAACGTGTTGCTGAACCAGATGTCCGACGCAGAACGCGACGCCCACATCGCCGCGTA
CCGCGCCGGCGGCGCAGTTCCTGCC
>162_prmA

(SEQ ID NO: 167)

GAGCTTGACGAAAGCACATGCGAAGATCACCGAACTGTCGTGGGAACCGACATTCGCGA
CTCCCGCGACACGATTCGGCACGGACTACACGTTCGAGAAGGCCCCGAAGAAGGACCCA
CTCAAGCAGATCATGCGGTCGTACTTCCCGATGGAAGAGGAGAAGGACAACCGCGTCTA
CGGCGCGATGGACGGCGCGATCCGCGGCAACATGTTCCGTCAGGTCCAGGAACGCTGGC
TGGAATGGCAGAAGCTGTTCCTGTCGATCATTCCGTTTCCCGAAATCTCGGCGGCGCGC
GCGATGCCGATGGCTATCGACGCCGTACCGAACCCGGAGATCCACAATGGGCTCGCGGT
GCAGATGATCGACGAGGTTCGTCACTCCACGATCCAGATGAACCTCAAGAAGCTGTACA
TGAACAATTACATCGACCCCGCCGGGTTCGACATCACCGAAAAGGCGTTCTCGAACAAC
TACGCGGGCACGATCGGCCGGCAATTCGGTGAAGGCTTCATCACCGGCGACGCGATCAC
CGCCGCCAACATCTACCTGACCGTCGTCGCGGAGACCGCGTTCACCAACACCCTGTTCG
TGGCCATGCCCGATGAAGCTGCAGCCAACGGCGACTACCTGTTGCCGACGGTGTTCCAC
TCGGTGCAGTCCGACGAATCCCGCCACATCTCCAACGGCTACTCGATCCTGCTCATGGC
GTTGGCCGACGAGCAGAACCGGCCGCTGCTCGAGCGCGACCTGCGGTACGCGTGGTGGA
ACAACCACTGCGTCGTCGATGCCGCGATCGGTACGTTCATCGAGTACGGCACGAAGGAC
CGCCGCAAGGATCGAGAGAGCTACGCCGAGATGTGGCGACGGTGGATCTACGACGACTA
CTACCGCAGCTACCTGTTGCCGCTCGAGAAGTACGGTCTGACGATCCCGCACGACCTGG
TCGAGGAGGCGTGGAAGCGGATCACCGAGAAGAGCTACGTGCACGAGGTCGCACGGTTC
TTCGCGACCGGCTGGCCCGTGAACTACTGGCGGATCGACGCGATGACCGACGCCGACTT
CGAATGGTTCGAAGACAAGTACCCGGGCTGGTACTCGAAGTTCGGCAAGTGGTGGGAGA
ACTACAACCGCCTCGCCTACCCGGGCCGGAACAAGCCGATCGCGTTCGAGGAAGTCGGC
TACCAGTACCCGCACCGCTGCTGGACGTGCATGGTGCCGGCCCTGGTCCGTGAGGACAT
GGTGGTCGAGAAGGTCGACGGACAGTGGCGCACCTACTGCTCGGAGCCGTGCTACTGGA
CCGACGCGGTCGCGTTCCGCGGTGAGTACGAGGGCCGGGAGACACCGAACATGGGTCGA
CTCACCGGGTTCCGCGAGTGGGAGACCCTCCACCACGACAAGGATCTCGCCGACATCGT
CTCGGATCTCGGCTATGTGCGCGACGACGGCAAGACTCTCATCGGGCAACCGCACCTCG
ATTTGAACCCGAAGAAGATGTGGACCCTCGACGACGTGCGGGGGAACACCTTCCAGAGT
CCGAACGTGTTGTTGAACCAGATGTCCGACGCACAGCGGGCAGCGCACATCGCGGAGTA
CCGCGCAGGCGCGACACCGCTG
>163_prmA

(SEQ ID NO: 168)

GAGCTTGACAAAAGCCCATGCGAAGATCACCGAGTTGTCCTGGGAGCCCACCTTCGCCA
CCCCGGCCACCCGGTTCGGTACCGACTACACATTCGAGAAGGCTCCCAAGAAGGATCCG
CTCAAGCAGATCATGCGGTCGTACTTCCCGATGGAGGAGGAAAAGGACAACCGCGTGTA
CGGCGCCATGGACGGCGCCATCCGCGGCAACATGTTCCGCCAGGTGCAGGAACGTTGGC
TGGAATGGCAGAAGCTGTTCCTGTCGATCATCCCGTTCCCCGAGATCTCTGCGGCCCGC
GCGATGCCGATGGCCATCGACGCCGTCCCCAATCCCGAGATCCACAATGGCCTGGCCGT
GCAGATGATCGACGAGGTTCGTCATTCGACGATCCAGATGAACCTCAAGAAGCTGTACA
TGAACAACTACATCGACCCGGCCGGCTTCGACATCACCGAGAAGGCGTTCGCCAACAAC
TACGCCGGCACCATCGGCCGCCAGTTCGGCGAGGGTTTCATCACCGGCGACGCCATCAC
CGCGGCCAACATCTACTTGACCGTCGTCGCCGAAACAGCCTTCACCAACACGCTTTTCG
TCGCCATGCCCGACGAGGCCGCCGCCAACGGCGACTACCTGCTGCCGACCGTGTTCCAC
TCCGTCCAGTCCGACGAGTCGCGACACATCTCCAACGGCTACTCGATCCTGCTCATGGC
ACTCGCCGACGAGCGCAACCGCCCCCTGCTGGAGCGCGACCTGCGCTACGCATGGTGGA
ACAATCACTGCGTCGTCGACGCCGCCATCGGCACGTTCATCGAGTACGGCACCAAGGAC
CGTCGCAAGGATCGCGAGAGCTACGCCGAGATGTGGCGTCGCTGGATCTACGACGACTA
CTACCGCAGTTACCTTCTGCCGCTGGAGAAGTACGGGCTCACCATCCCGCACGACCTTG
TCGAGGAGGCGTGGAACCGGATCACCAACAAGCACTACGTCCACGAGGTCGCCCGCTTC
TTCGCCACCGGCTGGCCGGTCAACTACTGGCGCATCGACGCCATGACCGACAAGGACTT
CGAGTGGTTCGAGCACAAATACCCCGGCTGGTACAACAAGTTCGGCAAGTGGTGGGAGA
ACTACAACCGGCTCGCCTACCCGGGCCGCAACAAGCCGATCGCCTTCGAAGAGGTCGGG
TACGAGTACCCGCACCGGTGCTGGACCTGCATGGTGCCCGCCCTCATCCGCGAGGACAT
GGTCACCGAGAAGGTCGACAACCAGTGGCGSACSTACTGCTCGGAGACCTGCTATTGGA
CCGATGCGGTGGCGTTCCGGGGCGAGTACGAGGGTCGTGAGACCCCGAACATGGGTCGC
CTCACCGGTTTCCGTGAATGGGAGACGCTCCATCACGGCAAGGATCTCGCCGACATCAT
CCAGGACCTGGGTTATGTCCGAGATGACGGCAAGACCTTGATCCCGCAGCCGCACCTCG
ATCTGGACCCGAAGAAGATGTGGACGCTCGACGATGTCCGCGGCAACGTCTTCAACAGC
CCGAACGTGCTGCTCAACGAGATGTCCGACGAGGAACGGGACGCCCACATCGCGGCGTA
CCGCGCCAACACCAACGGGGCCGTTCCGGCC
>167_prmA

(SEQ ID NO: 169)

AAGCCTGACAAAGGCCCACGCGAAAATCACCGAACTGTCATGGGATCCGACATTCGCAA
CCCCGGCCACCCGGTTCGGCACCGACTACACCTTCGAGAAGGCTCCGAAGAAGGATCCT
CTCAAACAGATCATGCGGTCCTACTTCCCGATGGAGGAAGAGAAGGACAACCGCGTGTA
CGGCGCCATGGACGGTGCCATCCGCGGCAACATGTTCCGCCAGGTGCAGCAGCGGTGGC
TCGAGTGGCAGAAGCTGTTCCTGTCGATCATTCCGTTCCCGGAGATCTCGGCAGCCCGA
GCGATGCCGTTGGCCATCGACGCCGTCCCCAACCCGGAAATCCACAACGGGCTGGCGGT
GCAGATGATCGACGAGGTTCGTCACTCGACGATCCAGATGAACCTCAAGAAGCTGTACA
TGAACAACTACATCGATCCCGCCGGTTTCGACATGACCGAGAAGGCGTTCGCGAACAAC
TACGCGGGCACCATCGGCAGGCAGTTCGGTGAAGGGTTCATCACCGGCGACGCGATCAC
CGCGGCGAACATCTATCTGACCGTGGTCGCCGAGACGGCGTTCACCAACACCCTGTTCG
TAGCCATGCCCGACGAGGCGGCCGCCAACGGTGACTACCTGTTGCCGACGGTCTTCCAC
TCGGTGCAGTCGGACGAGTCGCGGCACATCTCCAACGGCTACTCGATCCTGCTGATGGC
ACTCGCCGACGAGCGCAACCGTCCACTGCTCGAACGTGACCTGCGGTACGCGTGGTGGA
ACAACCACTGCGTCGTCGACGCCGCGATCGGCACCTTCATCGAGTACGGCACGAAGGAC
CGCCGCAAGGACCGGGAAAGTTACGCCGAGATGTGGCGTCGATGGATCTACGACGACTA
CTACCGCAGCTACCTCATCCCGCTCGAGAAGTACGGGCTGACGATCCCGCACGACCTGG
TCGAGGAGGCCTGGAAGCGGATCACCGACAAGGGCTACGTCCACGAGGTGGCCCGGTTC
TTCGCTACCGGATGGCCGGTGAACTACTGGCGGATCGACGCGATGACCGACAAGGACTT
CGAGTGGTTCGAGCACAAGTACCCGGGCTGGTACTCGAAGTACGGCAAGTGGTGGGAGG
AGTACAACCGGCTCGCCTACCCCGGCCGCAACAAACCGATCGCGTTCGAGGAGGTCGGG
TACCAGTACCCGCACCGGTGCTGGACCTGCATGGTTCCCGCCCTCATCCGTGAGGACAT
GGTCGTGGAGAAGGTCGACGACCAATGGCGGACCTACTGCTCGGAAACCTGCTACTGGA
CCGACGCCGTCGCGTTCCGCAGCGAGTACCAGGGCCGACCGACCCCGAACATGGGCCGG
CTCACCGGATTCCGGGAGTGGGAAACCCTGCACCACGGCAAGGACCTCGCCGACATCGT
CTCCGATCTCGGCTACGTCCGCGACGACGGCAAGACCCTGGTCGGCCAGCCGCACCTCG
ATCTGGACGATCCGAAGAAGATGTGGACTCTCGACGACGTGCGGGGCAACACCTTCCAG
AGCCCGAACGTGCTCTTGAACGAGATGTCCGACGCCGAACGCAACGCGCACATCGCCGC
GTACCGCGCCGGCGGCACAGTTCCGGCC
>168_prmA

(SEQ ID NO: 170)

AAGCCTGACAAAGGCCCACGCGAAGATCACCGAACTGTCATGGGATCCGACATTCGCAA
CCCCGGCCACCCGGTTCGGCACCGACTACACCTTCGAGAAGGCTCCGAAGAAGGATCCT
CTCAAGCAGATCATGCGGTCCTACTTCCCGATGGAGGAAGAGAAGGACAACCGCGTGTA
CGGCGCCATGGACGGTGCCATCCGCGGCAACATGTTCCGCCAGGTGCAGCAGCGGTGGC
TCGAATGGCAGAAGCTGTTCCTGTCGATCATTCCGTTCCCGGAGATCTCGGCCGCCCGA
GCGATGCCGATGGCCATCGACGCCGTCCCCAACCCGGAGATCCACAACGGGCTGGCGGT
GCAGATGATCGACGAGGTTCGTCACTCGACGATCCAGATGAACCTCAAGAAGCTTTACA
TGAACAACTACATCGATCCCGCCGGTTTCGACATGACCGAGAAGGCGTTCGCGAACAAC
TACGCGGGGACCATCGGCAGGCAGTTCGGTGAAGGGTTCATCACCGGCGACGCGATCAC
CGCGGCGAACATCTATCTGACCGTGGTCGCCGAGACGGCGTTCACCAACACCCTGTTCG
TTGCCATGCCCGACGAGGCGGCCGCCAACGGTGACTACCTGTTGCCGACGGTCTTCCAC
TCGGTGCAGTCGGACGAGTCGCGGCACATCTCCAACGGCTATTCGATCCTGCTGATGGC
ACTCGCCGACGAGCGCAACCGTCCACTACTCGAACGTGACCTGCGGTACGCGTGGTGGA
ACAACCACTGCGTCGTCGACGCCGCGATCGGCACCTTCATCGAGTACGGCACGAAGGAC
CGCCGCAAGGACCGGGAGAGTTACGCCGAGATGTGGCGGCGGTGGATCTACGACGATTA
CTACCGCAGCTACCTCATCCCGCTCGAGAAGTACGGCCTGACGATCCCGCACGACCTGG
TCGAGGAGGCGTGGAAGCGGATCACCGACAAGGGCTACGTCCACGAGGTGGCCCGGTTC
TTCGCCACCGGATGGCCGGTGAACTACTGGCGGATCGACGCGATGACCGACAAGGACTT
CGAGTGGTTCGAGCACAAGTACCCGGGCTGGTACTCGAAGTACGGCAAGTGGTGGGAGG
AGTACAACCGGCTCGCCTACCCCGGTCGCAACAAGCCGATCGCGTTCGAGGAGGTCGGA
TACCAGTACCCGCACCGGTGCTGGACCTGCATGGTTCCCGCACTCATCCGTGAGGACAT
GGTCGTGGAGAAGGTCGACGAGCAGTGGCGGACCTACTGCTCGGAAACCTGCTACTGGA
CCGACGCCGTCGCGTTCCGCAGCGAGTACCAGGGCCGCCCGACCCCGAACATGGGCCGG
CTCACGGGATTCCGGGAGTGGGAAACCCTGCATCACGGCAAGGACCTCGCCGACATCGT
CTCCGATCTCGGCTACGTCCGCGACGACGGCAAGACCCTGGTCGGTCAGCCGCACCTCG
ATCTGGACGATCCGAAGAAGATGTGGACTCTCGACGACGTGCGGGGCAACACCTTCCAG
AGCCCGAACGTGCTCTTGAACGAGATGTCCGACGCCGACCGCAACGCGCACATCGCCGC
GTACCGCGCCGGCGGCGCAGTTCCGGCC
>170_prmA

(SEQ ID NO: 171)

GAGCCTGACAAAGGCCCACGCGAAGATCAGCGAGTTGACCTGGGATCCGACATTCGCAA
CCCCGGCTACCCGATTCGGCACCGATTACACGTTCGAGAAGGCTCCGAAGAAGGACCCT
CTCAAACAGATCATGCGGTCATACTTCCCGATGGAGGAAGAGAAGGACAACAGGGTCTA
CGGCGCTATGGACGGCGCGATCCGCGGCAATATGTTCCGCCAGGTCCAACAGCGTTGGA
TGGAGTGGCAGAAGCTGTTCCTGTCGATCATTCCGTTCCCGGAGATCTCCGCCGCCAGG
GCTATGCCGATGGCCATCGACGCCGTGCCGAACCCGGAAATTCACAACGGTTTGGCGGT
CCAGATGATCGACGAGGTACGGCACTCGACGATTCAGATGAATCTCAAGAAGCTCTACA
TGAACAACTACATCGACCCGGCCGGGTTCGACATGACCGAGAAGGCGTTCGCGAACAAC
TACGCGGGCACGATCGGCCGGCAGTTCGGTGAAGGTTTCATCACCGGCGACGCGATCAC
GGCGGCCAATATCTATCTGACGGTTGTCGCGGAGACGGCGTTCACGAACACACTGTTCG
TCGCGATGCCAGACGAAGCCGCCGCAAACGGTGATTACCTGCTGCCCACCGTGTTTCAC
TCGGTGCAGTCTGACGAGTCGCGGCACATCTCCAACGGTTATTCGATTCTGTTGATGGC
CCTGGCCGACGAGCGTAACCGTCCGCTGCTCGAGCGAGATCTGCGCTACGCGTGGTGGA
ACAACCACTGTGTCGTGGACGCCGCGATCGGCACGTTCATCGAATACGGCACCAAGGAC
CGCCGCAAGGACCGCGAGAGCTACGCCGAGATGTGGCGTCGGTGGATCTACGACGACTA
CTACCGCAGCTACCTGATTCCGTTGGAGAAGTACGGCCTGACCATCCCGCACGATCTGG
TCGAGGAAGCCTGGAATCGCATCACGAACAAGGGATACGTGCACGAGGTTGCGCGCTTC
TTCGCAACAGGATGGCCGGTCAACTACTGGCGGATCGACACGATGACCGACAAGGACTT
CGAGTGGTTCGAGCACAAGTATCCCGGTTGGTACTCGAAGTACGGCAAGTGGTGGGAGG
AGTACAACCGCCTCGCTTACCCCGGCCGGAACAAGCCGATCGCATTCGAGGAAGTGGGA
TACCAGTACCCGCATCGGTGCTGGACCTGCATGGTGCCTGCGCTCATTCGTGAAGACAT
GGTTGTGGAGAAGGTCGACAACCAGTGGCGAACCTACTGCTCGGAAACGTGCTACTGGA
CCGACGCGGTGGCCTTCCGTGAGGAGTATCAGGGCAGGCCGACGCCGAACATGGGTCGG
CTCACCGGATTTCGTGAGTGGGAAACCCTGCACCACGACAAGGATCTCGCGGACATCGT
CAAAGACCTCGGTTACGTCCGAGACGACGGGAAGACCCTGGTCGGCCAGCCGCATCTGC
ACCTGGACGACCCGAAGAAGCTGTGGACTCTCGACGACGTTCGTGGCAACACGTTCATG
AGCCCGAATGTGCTCTTGAACCAGATGTCCGACGCCGAACGCATCGCCCATATCGCGGA
ATACCGCGCCGGGGCGACTCCGGCC
>171_prmA

(SEQ ID NO: 172)

AAGCCTGACAAAGGCCCACGCGAAGATCACCGAACTGTCATGGGATCCGACATTCGCAA
CCCCGGCCACCCGGTTCGGAACCGACTACACCTTCGAGAAGGCCCCCAAGAAAGACCCT
CTCAAGCAGATCATGCGGTCCTACTTCCCGATGGAGGAGGAAAAAGACAACCGCGTGTA
CGGCGCCATGGACGGTGCGATCCGCGGCAACATGTTCCGGCAGGTGCAGCAGCGGTGGC
TCGAATGGCAGAAGCTGTTCCTGTCGATCATTCCGTTCCCGGAGATCTCGGCCGCCCGA
GCGATGCCGATGGCCATCGACGCCGTGCCCAACCCGGAAATCCACAACGGGCTTGCGGT
ACAGATGATCGACGAAGTTCGTCACTCGACGATCCAGATGAACCTCAAGAAGTTGTACA
TGAACAACTACATCGATCCCGCCGGGTTCGACATGACGGAGAAGGCGTTCGCGAACAAC
TACGCGGGCACCATCGGCCGGCAGTTCGGTGAAGGGTTCATCACCGGCGACGCGATCAC
CTCGGCGAACATCTACCTGACCGTGGTCGCCGAAACCGCGTTCACCAACACCCTGTTCG
TGGCCATGCCCGACGAGGCCGCCGCCAACGGCGACTACCTGTTGCCGACGGTCTTCCAC
TCGGTGCAGTCGGACGAGTCGCGGCACATCTCCAACGGTTACTCGATCCTGCTGATGGC
CCTCGCCGACGAGCGAAACCGTCCACTGCTCGAACGCGATCTGCGGTACGCGTGGTGGA
ACAACCACTGCGTCGTCGACGCCGCGATCGGCACCTTCATCGAGTACGGCACCAAGGAC
CGCCGCAAGGACCGGGAGAGCTACGCCGAGATGTGGCGGCGGTGGATTTACGACGACTA
CTACCGCAGCTACCTCATCCCGCTCGAGAAGTACGGTCTGACGATTCCGCACGATCTGG
TCGAGGAGGCGTGGAAGCGGATCACCGAAAAGGGTTACGTCCACGAGGTGGCACGGTTC
TTCGCCACCGGCTGGCCGGTGAACTACTGGCGGATCGATGCGATGACCGACAAGGACTT
CGAGTGGTTCGAACACAAGTACCCGGGCTGGTACTCGAAGTACGGCAAGTGGTGGGAGG
AGTACAACCGGCTCGCCTACCCCGGCCGCAACAAGCCGATCGCATTCGAAGAGGTCGGG
TACCAGTACCCGCACCGGTGCTGGACCTGCATGGTGCCCGCCCTCATCCGCGAAGACAT
GGTCGTGGAGAAGGTGGACAACCAGTGGCGGACCTACTGCTCGGAAACCTGCTACTGGA
CCGACGCCGTCGCGTTCCGCGAGGAGTATCAGGGTAAGCCGACCCCGAATATGGGACGA
CTCACCGGGTTCCGTGAATGGGAGACCCTGCACCACGGCAAGGACCTCGCCGACATCGT
CTCCGACCTGGGGTACGTCCGCGACGACGGCAAGACCCTCGTCGGTCAGCCGCACCTCG
ATTTGGACGACCCGAAGAAGATGTGGACCCTCGACGATGTGCGGGGCAACACCTTCCAG
AGCCCGAACGTGCTCTTGAACCAGATGTCCGACGCCGAACGCGACGCCCACATCGCCGC
ATACCGCGCAGGCAGAACCGTTCCTGCG
>172_prmA

(SEQ ID NO: 173)

AAGCCTGACAAAGGCCCACGCGAAGATCACCGAACTGTCATGGGATCCGACCTTCGCGA
CCCCGGCCACCCGGTTCGGCACCGACTACACCTTCGAGAAGGCTCCGAAGAAGGACCCT
CTCAAGCAGATCATGCGGTCCTACTTCCCGATGGAGGAAGAGAAGGACAACCGCGTGTA
CGGCGCCATGGACGGTGCCATCCGCGGCAACATGTTCCGCCAGGTGCAGCAGCGGTGGC
TCGAATGGCAGAAGCTGTTCCTGTCGATCATCCCGTTCCCGGAGATCTCAGCGGCCCGT
GCGATGCCGATGGCTATCGACGCCGTGCCCAACCCGGAAATTCACAACGGGCTCGCGGT
GCAGATGATCGACGAGGTTCGTCACTCGACGATCCAGATGAACCTCAAGAAGCTGTACA
TGAACAACTACATCGACCCGGCCGGGTTCGACATCACCGAGAAGGCGTTCTCGAACAAC
TACGCCGGCACCATCGGCCGACAGTTCGGTGAAGGCTTCATCACCGGTGACGCGATCAC
CGCCGCGAACATCTACCTGACCGTGGTCGCCGAGACCGCGTTCACGAACACCCTGTTCG
TCGCGATGCCCGACGAGGCCGCCGCCAATGGTGACTACCTGCTGCCGACGGTGTTCCAC
TCGGTGCAGTCCGACGAGTCCCGGCACATCTCCAACGGCTATTCGATCCTGCTGATGGC
CCTCGCCGACGAGCGCAACCGGCCGCTGCTCGAACGAGACCTGCGGTACGCGTGGTGGA
ACAACCACTGTGTCGTCGACGCCGCGATCGGCACCTTCATCGAGTACGGCACCAAGGAC
CGCCGCAAGGACCGGGAAAGTTACGCCGAGATGTGGCGGCGGTGGATCTACGACGACTA
CTACCGCAGCTACCTCATCCCCCTCGAGAAGTACGGGCTGACGATTCCGCACGACCTGG
TCGAGGAGTCGTGGAAGCGCATCACCGAGAAGGGTTACGTCCACGAGGTAGCCCGGTTC
TTCGCGACCGGGTGGCCGGTGAACTACTGGCGGATCGACACGATGACCGACAAGGACTT
CGAGTGGTTCGAGCACAAGTACCCGGGCTGGTACTCGAAGTACGGCAAGTGGTGGGAGG
AGTACAACCGTCTCGCCTACCCGGGCCGCAACAAGCCGATTGCGTTCGAGGAGGTCGGG
TACCAGTACCCGCACCGGTGCTGGACGTGCATGGTTCCGGCCCTGATCCGCGAGGACAT
GGTGGTCGAGAAGGTGGACAACCAGTGGCGGACCTACTGCTCGGAGACCTGCTACTGGA
CCGACGCAGTCGCCTTCCGCGGTGAGTACGAGGGCCGGGAAACCCCGAACATGGGACGT
CTCACCGGATTCCGCGAGTGGGAGACGTTGCATCACGGCAAGGATCTGGCCGACATCGT
GCAGGACCTGGGTTATGTCCGCGACGACGGTAAGACCCTCATCGGTCAGCCGCACCTGC
ACCTGGACGATCCGAAGAAGATGTGGACCCTCGATGACGTGCGGGGCAACACCTTCCAG
AGTCCGAACGTGCTGCTGAACCAGATGTCGGACGCCGAACGCAACGCCCACATTGCCGC
GTACCGCGCCGGCGGCGCAGTTCCGGCC
>173_prmA

(SEQ ID NO: 174)

GAGCCTGACCAAGGCCCATGCGAAGATCACCGAGCTGTCGTGGGAACCGACGTTCGCGA
CGCCGGCCACCCGCTTCGGCACCGACTACACGTTCGAGAAGGCCCCCAAGAAGGACCCT
CTCAAGCAGATCATGCGGTCCTACTTCCCCATGGAGGAGGAGAAGGACAACCGGGTCTA
CGGCGCGATGGACGGCGCCATCCGCGGGAACATGTTCCGGCAGGTCCAGCAGCGCTGGC
TGGAGTGGCAGAAGCTGTTCCTCTCGATCATCCCGTTCCCGGAGATCTCGGCGGCCCGC
GCGATGCCGATGGCCATCGACGCGGTGCCCAACCCCGAGATCCACAACGGGCTCGCCGT
GCAGATGATCGACGAGGTTCGTCACTCGACGATCCAGATGAACCTCAAGAAGCTCTACA
TGAACAACTACATCGACCCCGCCGGGTTCGACATGACCGAGAAGGCGTTCGCGAACAAC
TACGCGGGCACCATCGGCCGGCAGTTCGGGGAGGGCTTCATCACCGGTGACGCGATCAC
CGCGGCCAACATCTACCTGACCGTGGTCGCGGAGACGGCCTTCACGAACACCCTGTTCG
TGGCGATGCCCGACGAGGCGGCCGCCAACGGCGACTACCTGCTGCCCACCGTGTTCCAT
TCGGTGCAGTCCGACGAGTCGCGGCACATCTCCAACGGCTACTCGATCCTGCTCATGGC
GCTGGCCGACGAGCGGAACCGGCCGCTGCTCGAGCGGGACCTGCGCTACGCGTGGTGGA
ACAACCACTGCGTGGTCGACGCCGCGATCGGCACCTTCATCGAGTACGGCACCAAGGAC
CGCCGCAAGGATCGCGAGAGCTACGCCGAGATGTGGCGGCGGTGGATCTACGACGACTA
CTACCGCAGCTACCTCATCCCGCTCGAGAAGTACGGGCTGACCATTCCGCACGACCTCG
TCGAGGAGGCGTGGAAGCGCATCACCGAGAAGGGCTACGTCCACGAGGTGGCCAGGTTC
TTCGCGACGGGCTGGCCGGTGAACTACTGGCGGATCGACGCCATGACCGACGCGGACTT
CGAGTGGTTCGAGCACAAGTACCCGGGCTGGTATTCCAAGTACGGCAAGTGGTGGGAGA
ACTACAACCGCCTCGCCTACCCCGGCCGCAACAAGCCGATCGCGTTCGAGGAGGTGGGA
TACCAGTACCCGCACCGCTGCTGGACGTGCATGGTGCCCGCCCTCATCCGCGAGGACAT
GGTCGTGGAGAAGGTGGACGACCAGTGGCGGACCTACTGCTCGGAGACCTGCTACTGGA
CCGACGCCGTCGCGTTCCGCAGCGAGTACGAGGGACGTCCCACCCCGAACATGGGCCGC
CTCACCGGTTTCCGTGAATGGGAGACCCTGCACCACGACAAGGATCTCGCCGACATCGT
GCAGGACCTCGGGTACGTGCGCGACGACGGCAAGACCCTCGTCGGTCAGCCGCATCTCG
ACCTCGACCCGAAGAAGATGTGGACCCTCGACGACGTGCGGGGCAACACCTTCCAGAGC
CCGAACGTGTTGCTGAACCAGATGTCCGACGCAGAACGCGACGCCCACATCGCCGCGTA
CCGCGCCGGCGGCGCAGTTCCTGCC
>174_prmA

(SEQ ID NO: 175)

AAGCCTGACAAAGGCCCACGCGAAGATCACCGAACTGTCATGGGATCCGACATTCGCCA
CCCCGGCCACCCGGTTCGGCACCGACTACACCTTCGAGAAGGCTCCGAAGAAGGACCCT
CTCAAGCAGATCATGCGGTCCTACTTCCCGATGGAGGAAGAGAAGGACAACCGCGTGTA
CGGCGCCATGGACGGTGCCATCCGCGGCAACATGTTCCGCCAGGTGCAGCAGCGGTGGC
TCGAATGGCAGAAGCTGTTCCTGTCGATCATTCCGTTCCCGGAGATCTCGGCGGCCCGA
GCGATGCCGATGGCCATCGACGCCGTCCCCAACCCGGAAATCCACAACGGGCTGGCGGT
GCAGATGATCGACGAGGTTCGTCACTCGACGATCCAGATGAACCTCAAGAAGCTGTACA
TGAACAACTACATCGATCCCGCCGGGTTCGACATCACCGAGAAGGCGTTCTCGAACAAC
TACGCGGGCACCATCGGCCGGCAGTTCGGCGAAGGGTTCATCACCGGTGACGCAATCAC
CGCCGCGAACATCTACCTGACCGTGGTCGCCGAGACCGCGTTCACCAACACCCTGTTCG
TCGCGATGCCCGACGAGGCCGCCGCCAACGGTGACTACCTGCTGCCGACGGTGTTCCAC
TCGGTGCAATCCGACGAGTCCCGGCACATCTCCAACGGCTATTCGATCCTGCTGATGGC
GCTCGCCGACGAGCGCAACCGGCCTCTGCTCGAACGGGATCTGCGGTACGCATGGTGGA
ACAACCACTGTGTCGTCGACGCAGCGATCGGCACCTTCATCGAGTACGGCACGAAGGAC
CGCCGCAAGGACCGCGAAAGTTACGCCGAGATGTGGCGGCGGTGGATCTACGACGACTA
CTACCGCAGCTACCTCATCCCGCTCGAGAAGTACGGCCTGACGATCCCGCACGACCTGG
TCGAGGAGTCGTGGAAGCGGATCACCGAGAAGGGCTACGTCCACGAGGTGGCCCGGTTC
TTCGCCACCGGCTGGCCGGTGAACTACTGGCGGATCGACACGATGACCGACAAGGACTT
CGAATGGTTCGAGCACAAGTACCCCGGCTGGTACTCGAAGTACGGCAAATGGTGGGAGG
AGTACAACCGCCTCGCCTACCCCGGCCGTAACAAGCCGATCGCGTTCGAGGAGGTCGGG
TACCAGTACCCGCACCGGTGCTGGACCTGCATGGTGCCGGCCCTGATCCGCGAGGACAT
GGTCGTGGAGAAGGTCGACGACCAGTGGCGGACCTACTGCTCGGAGACTTGCTACTGGA
CCGACGCGGTCGCGTTCCGCAGCGAGTACGAGGGCCGGGATACCCCGAATATGGGGCGT
CTCACCGGATTCCGGGAGTGGGAGACCCTCCATCACGGCAAGGATCTCGCTGACATCGT
GCAGGACCTCGGTTACGTGCGCGACGACGGTAAGACCCTCATCGGTCAGCCGCACCTCC
ATCTGGACGACCCGAAGAAGATGTGGACTCTGGACGACGTACGAGGCAACACCTTCCAG
AGTCCGAACGTGCTGCTGAACCAGATGTCCGACGCCGAACGCAACGCGCACATCGCCGC
GTACCGCGCCGGCGGCACAGTTCCGGCC

TABLE 5

>048_prmD

(SEQ ID NO: 176)

CGGCGTCACCCTGATGAACACGCCCATCGGCCGCGTCGTCGCCGACGTCATGGGCGCCA
AGGAGGGTGTCGAACTCACCGAGTACCCGTCGATGATCCGCGTCGACGGCGTCAACCGG
CTCGAGTTCGACTACGACGAGCTCACCGACGCCCTGGGTCAGGAGTTCGACGGATCGGT
CTTCGAGGAGATCAGCTCCACCCACTACGGGCGAATGGTGCACCTCGACGACCGGACCT
TCCTGTTCGCGAGCCCCGAG
>049_prmD

(SEQ ID NO: 177)

GTGAGCATGCAATTCGGATCGGCCACCGAGTTCTCCAACATGTGTGGCGTCACCCTGAT
GAACACCCCGATCGGCCGCGTGGTCGCCGAGGTCATGGGCGCCAAGGACGGCGTGCAGC
TGACGGAGTACCCGTCGATGATCCGCGTCGACGGCGTCAACCGCCTCGAGTTCGACTAC
GAGGAACTTACCGACGCTCTCGGCTCCGACTTCGACGGCTCCGTCTTCGAGGAGATCAG
CTCCACCCACTACGGGCGCATGGTGCACCTCGATGACAAGACCATGCTCTTCGCCAGTC
CCGAG
>052_prmD

(SEQ ID NO: 178)

GTGAGCATGCAATTCGGATCGTCCACCGAGTTCTCCAACATGTGTGGCGTCACCCTGAT
GAACACCCCGATCGGCCGCGTGGTCGCCGAGGTCATGGGCGCCAAGGACGGCGTGCAGC
TGACGGAGTACCCGTCGATGATCCGCGTCGACGGCGTCAACCGCCTCGAGTTCGACTAC
GAGGAACTCACCGACGCTCTCGGCTCCGACTTCGACGGCTCCGTCTTCGAGGAGATCAG
CTCCACCCACTACGGGCGCATGGTGCACCTCGATGACAAGACCATGCTCTTCGCCAGTC
CCGAG
>105_prmD

(SEQ ID NO: 179)

GTGAGCATGCAATTCGGATCGTCCACCGAGTTCTCCAACATGTGTGGCGTCACCCTGAT
GAACACCCCGATCGGCCGCGTGGTCGCCGAGGTCATGGGCGCCAAGGACGGCGTGCAGC
TGACGGAGTACCCGTCGATGATCCGCGTCGACGGCGTCAACCGCCTCGAGTTCGACTAC
GAGGAACTCACCGACGCTCTCGGCTCCGACTTCGACGGCTCCGTCTTCGAGGAGATCAG
CTCCACCCACTACGGGCGCATGGTGCACCTCGATGACAAGACCATGCTCTTCGCCAGTC
CCGAGGACGCCGCCGAGTACATCGGATTCGATCTCACGGCGCAC
>106_prmD

(SEQ ID NO: 180)

GTGAGCATGCAATTCGGATCGGCCACCGAGTTCTCCAACATGTGTGGCGTCACCCTGAT
GAACACCCCGATCGGACGCGTCGTCGCCGACGTCATGGGCGCCAAGGAGGGAGTGGAGC
TGACGGAGTACCCGTCGATGATCCGCGTCGACGGCGTGAACCGCCTCGAATTCGACTAC
GCCGAGCTCACCGACGCCCTCGGTGAGGACTTCGACGGATCGATCTTCGAGGAGATCAG
CTCCACCCACTACGGGCGCATGGTGCATCTCGACGACAAGACCATGCTCTTCGCCAGTC
CCGAGGACGCCGCCGAGTACATCGGATTCGATCTCACGGCGCAC
>152_prmD

(SEQ ID NO: 181)

TGGCGTCACCTTGATGAACACCCCCATCGGCCGTGTCGTCGCGGAGGTGATGGGCGCCA
AGGACGGCGTGGAGCTGACCGAGTACCCGTCGATGATCCGCGTCGACGGCCAACGCCTG
CTCGACTTCGACTACGAGGAACTCACCGACGCCCTGGGTCAGGAGTTCGACGGCTCCAT
CTTCGAGGAGATCAGCTCCACCCACTACGGGCGCATGGTCCACCTCGACGAGAAGACCC
TGCTGTTCGCCAGCCCGGAGGACGCCGCCGAGTACATCGGATTCGACCTCACGGCGCAG
>153_prmD

(SEQ ID NO: 182)

TGGCGTCACCTTGATGAACACCCCCATCGGCCGTGTCGTCGCGGAGGTGATGGGCGCCA
AGGACGGCGTGGAGCTGACCGAGTACCCGTCGATGATCCGCGTCGACGGCCAACGCCTG
CTCGACTTCGACTACGAGGAACTCACCGACGCCCTGGGTCAGGAGTTCGACGGCTCCAT
CTTCGAGGAGATCAGCTCCACCCACTACGGGCGCATGGTCCACCTCGACGAGAAGACCC
TGCTGTTCGCCAGCCCGGAG
>154_prmD

(SEQ ID NO: 183)

TGGCGTCACCTTGATGAACACCCCCATCGGCCGTGTCGTCGCGGAGGTGATGGGCGCCA
AGGACGGCGTGGAGCTGACCGAGTACCCGTCGATGATCCGCGTCGACGGCCAACGCCTG
CTCGACTTCGACTACGAGGAACTCACCGACGCCCTGGGTCAGGAGTTCGACGGCTCCAT
CTTCGAGGAGATCAGCTCCACCCATTACGGGCGCATGGTCCACCTCGACGAGAAGACCC
TGCTGTTCGCCAGCCCGGAG
>155_prmD

(SEQ ID NO: 184)

GTGAGCATGCAATTCGGATCGTCCACCGAGTTCTCCAACATGTGTGGCGTCACCTTGAT
GAACACCCCCATCGGCCGTGTCGTCGCGGAGGTGATGGGCGCCAAGGACGGCGTGGAGC
TGACCGAGTACCCGTCGATGATCCGCGTCGACGGCCAACGCCTGCTCGACTTCGACTAC
GAGGAACTCACCGACGCCCTGGGTCAGGAGTTCGACGGCTCCATCTTCGAGGAGATCAG
CTCCACCCACTACGGGCGCATGGTCCACCTCGACGAGAAGACCCTGCTGTTCGCCAGCC
CGGAG
>156_prmD

(SEQ ID NO: 185)

GTGACCATGCAATTCGGATCGACCACCGAGTTCTCCAACATGTGTGGCGTCACCTTGAT
GAACACCCCCATCGGCCGCGTCGTCGCGGAGGTGATGGGCGCCAAGGACGGTGTCGAGC
TGACCGAGTACCCGTCGATGATCCGCGTCGACGGCCAGAAGCTGCTGAATTTCGACTAC
GAGGAACTCACCGACGCTCTCGGTGAGGAGTTCGACGGCTCCATCTTCGAGGAGATCAG
CTCCACCCATTACGGGCGCATGGTTCACCTCGACGACAAGACCCTGCTGTTCGCCAGCC
CCGAAGACGCCGCCGAGTACATCGGATTCGACCTCACCGAGCAC
>157_prmD

(SEQ ID NO: 186)

TGGCGTCACCTTGATGAACACCCCCATCGGCCGTGTCGTCGCGGAGGTGATGGGCGCCA
AGGACGGCGTGGAGCTGACCGAGTACCCGTCGATGATCCGCGTCGACGGCCAACGCCTG
CTCGACTTCGACTACGAGGAACTCACCGACGCCCTGGGTCAGGAGTTCGACGGCTCCAT
CTTCGAGGAGATCAGCTCCACCCACTACGGGCGCATGGTCCACCTCGACGAGAAGACCC
TGCTGTTCGCCAGCCCGGAG
>158_prmD

(SEQ ID NO: 187)

CGGCGTCACGCTGATGAACACCCCCATCGGGCGGGTGGTCGCCGACGTGATGGGCGCCA
AGGACGGCGTGGAGCTCACCGAGTACCCGTCGATGATCCGGGTGGACGGCACCCGGCTC
ATCGAGTTCGACTACGCCGAGCTGACCGACGCGCTCGGTCAGGACTTCGACGGGTCCAT
CTTCGAGGAGATCAGTTCCACGCACTACGGCCGCATGGTGCACCTCGACGACAAGACCA
TGCTGTTCGCCAGCCCCGAG
>160_prmD

(SEQ ID NO: 188)

TGGCGTCACCTTGATGAACACCCCCATCGGCCGTGTCGTCGCGGAGGTGATGGGCGCCA
AGGACGGCGTGGAGCTGACCGAGTACCCGTCGATGATCCGCGTCGACGGCCAACGCCTG
CTCGACTTCGACTACGAGGAACTCACCGACGCCCTGGGTCAGGAGTTCGACGGCTCCAT
CTTCGAGGAGATCAGCTCCACCCACTACGGGCGCATGGTCCACCTCGACGAGAAGACCC
TGCTGTTCGCCAGCCCGGAG
>161_prmD

(SEQ ID NO: 189)

TGGCGTCACCCTGATGAACACCCCGATCGGCCGCGTGGTCGCCGAGGTCATGGGCGCCA
AGGACGGCGTGCAGCTGACGGAGTACCCGTCGATGATCCGCGTCGACGGCGTCAACCGC
CTCGAGTTCGACTACGAGGAACTCACCGACGCTCTCGGCTCCGACTTCGACGGCTCCGT
CTTCGAGGAGATCAGCTCCACCCACTACGGGCGCATGGTGCACCTCGATGACAAGACCA
TGCTCTTCGCCAGTCCCGAG
>162_prmD

(SEQ ID NO: 190)

CGGTGTCACGTTGATGAACACGCCGATCGGTCGTGTCGTCGCCGATGTCATGGGCACCA
AGGACGGTGTGGAGCTGACGGAGTATCCGTCGATGATCCGCGTCGACGGCACGAAGTTG
CTCGAATTCGACTACGACGAACTCACCGACGCTCTCGGCTCCGAGTTCGACGGATCGGT
GTTCGAGGAGATCAGCTCGACCCACTACGGACGCATGGTACATCTCGACGACAAGACGA
TGCTCTTCGCCAGCCCCGAA
>163_prmD

(SEQ ID NO: 191)

CGGCGTGACGCTGATGAACACCCCGATCGGCCGCGTCGTCGCCGACGTCATGGGTTCGA
AGGACGGGGTCGAACTCACCGAGTACCCGTCGATGATCCGCGTGGACGGGGTCAACCGA
CTCGAATTCGACTACGACGAGCTGACCGACGCACTCGGCCAGGACTTCGACGGATCGAT
CTTCGAGGAGATCAGCTCGACCCACTACGGGCGGATGGTGCACCTCGACGACCGGACCT
TCCTGTTCGCCAGCCCGGAG
>164_prmD

(SEQ ID NO: 192)

CGGTGTCACGTTGATGAACACCCCGATCGGCCGGGTCGTCGCGGAGGTGATGGGCGCGA
AGGACGGTGTGGAGCTGACCGAGTACCCGTCGATGATCCGCGTCGACGGCCAGAGGCTG
CTCGACTTCGACTACGACGAACTGACCGACGCCCTGGGGCAGGATTTCGACGGCTCGAT
CTTCGAGGAGATCAGCTCCACCCACTACGGGCGCATGGTCCACCTCGACGAGAAGACCC
TGCTGTTCGCAAGCCCCGAG
>167_prmD

(SEQ ID NO: 193)

TGGCGTGACCTTGATGAACACCCCCATCGGCCGTGTCGTCGCGGAGGTGATGGGCGCCA
AGGACGGTGTGGAGCTGACCGAGTACCCGTCGATGATCCGCGTCGACGGCCAGCGCCTG
CTCGACTTCGACTACGAGGAACTCACCGACGCCCTCGGCCAGGAATTCGACGGCTCCAT
CTTCGAGGAGATCAGCTCCACCCACTACGGGCGCATGGTCCACCTCGACGAGAAGACCC
TGCTGTTCGCCAGCCCCGAG
>168_prmD

(SEQ ID NO: 194)

TGGCGTCACCTTGATGAACACCCCCATCGGCCGTGTCGTCGCGGAGGTGATGGGCGCCA
AGGACGGCGTGGAGCTGACCGAGTACCCGTCGATGATCCGCGTCGACGGCCAACGCCTG
CTCGACTTCGACTACGGGGAACTCACCGACGCCCTGGGTCAGGAGTTCGACGGCTCCAT
CTTCGAGGAGATCAGCTCCACCCACTACGGGCGCATGGTCCACCTCGACGAGAAGACCC
TGCTGTTCGCCAGCCCGGAG
>170_prmD

(SEQ ID NO: 195)

TGGTGTGACCCTGATGAATACTCCGACGGGCCGCATCGTCGCGGAGGTGATGGGAGCCA
AGGACGGTGTCGAACTCACCGAGTATCCCTCGATGATTCGCGTGGACGGCAAACGCCTT
CTCAACTTCGACTACGAAGAGCTCACCGACGCACTGGGTTCGGAATTCGACGGCTCCAT
TTTCGAGGAGATCAGCTCCACCCACTACGGACGCATGGTTCATCTCGACGACAAGACAA
TGCTGTTCGCCAGTCCGGAA
>171_prmD

(SEQ ID NO: 196)

TGGCGTCACCCTGATGAACACCCCGACCGGTCGCGTCGTCGCCGAAGTCATGGGCGRCA
AGGACGGCGTGGAGCTGACCGARTAYCCMTCGATGATCCGCGTCGACGGCCAGARSCTG
CTCAACTTCGACTACGAGGAACTCACCGACGCCCTSGGYGAGGAATTCGACGGCTCCAT
CTTCGAGGAGATCAGCTCCACCCACTACGGACGCATGGTCCACCTCGACGACAAGACCA
TGCTGTTCGCCAGCCCCGAG

1. DNA sequences deduced from the chromosomal DNA of propane-oxidizing bacteria, comprising the gene prmA encoding the alpha subunit of the propane monooxygenase enzyme, characterized by the nucleotide sequences indicated in Table 4.

2. DNA sequences deduced from the chromosomal DNA of propane-oxidizing bacteria comprising the gene prmD encoding an ancillary protein involved in the oxidation reaction of propane, characterized by the nucleotide sequences indicated in Table 5.

3. An oligonucleotide complementary to the sequences of the gene prmA of propane-oxidizing bacteria according to claim 1, selected from the following sequences of forward and reverse primers for prmA:
FORWARD PRIMERS:
(SEQ ID NO: 1)
prmA_1F: CTTCCCGATGGARGARGARAARGA
(SEQ ID NO: 2)
XA_0301F: GCCCATGCGAAGATCACCGA
(SEQ ID NO: 3)
XA_0358F: CCGCTTCGGCACCGACTACAC
(SEQ ID NO: 4)
XA_0370F: ACCGACTACACCTTCGAGAAGGC
(SEQ ID NO: 5)
XA_0382F: TTCGAGAAGGCCCCCAAGAAGGA
(SEQ ID NO: 6)
XA_0406F: CCTCTCAAGCAGATCATGCGGTC
(SEQ ID NO: 7)
XA_0930F: ACGGTCTTCCACTCGGTGCAGTC
(SEQ ID NO: 8)
XA_0993F: TGATGGCGCTCGCCGACGAGCG
(SEQ ID NO: 9)
XA_1041F: CTGCGGTACGCGTGGTGGAACAA
(SEQ ID NO: 10)
XA_1089F: GCACCTTCATCGAGTACGGCAC
(SEQ ID NO: 11)
XA_1107F: CGGCACCAAGGACCGCCGCAAGGA
(SEQ ID NO: 12)
XA_1152F: GGCGGCGGTGGATCTACGACGA
(SEQ ID NO: 13)
XA_1170F: TCATCCCGCTCGAGAAGTACGG
(SEQ ID NO: 14)
XA_1233F: GTCGAGGAGGCGTGGAAGCG
(SEQ ID NO: 15)
XA_1305F: GGCTGGCCGGTGAACTACTGGCG
(SEQ ID NO: 16)
XA_1390F: TCCAAGTACGGCAAGTGGTGGGAG
(SEQ ID NO: 17)
XA_1485F: ACCGGTGCTGGACCTGCATGGT
(SEQ ID NO: 18)
XA_1625F: GGCCGCCCGACCCCGAACATGGG
(SEQ ID NO: 19)
XA_460F: GTGTACGGCGCCATGGACGG
(SEQ ID NO: 20)
XA_526F: CTCGAATGGCAGAAGCTGTTCCT
(SEQ ID NO: 21)
XA_586F: GCGATGCCGATGGCCATCGACGC
(SEQ ID NO: 22)
XA_745F: AAGGCGTTCGCGAACAACTACGC
(SEQ ID NO: 23)
XA_789F: TTCGGTGAAGGCTTCATCACCGG
(SEQ ID NO: 24)
prmA_2F: GGTCGCCGAGACNGCNTTYACNAA
(SEQ ID NO: 25)
prmA_49F: GCGAAGATCACCGAGCTGT
(SEQ ID NO: 26)
prmA_733(f): CGCAATCGTCCGCTGCTC
(SEQ ID NO: 27)
XA_16F: GGCGCACATTGAGTAGGCA
(SEQ ID NO: 28)
XA_17F: TGCAGATGATCGACGAGGT
(SEQ ID NO: 29)
XA_18F: TCGCGGCACATCTCCAACGG
(SEQ ID NO: 30)
XA_19F: CGGACTTCGAGTGGTTCGA
(SEQ ID NO: 31)
XA_20Rf: AACAAGCCGATCGCGTTCG
(SEQ ID NO: 32)
XA_21Rf: CCGAACATGGGCCGGCTCA
(SEQ ID NO: 33)
XA_22F: GCCCGACCCCGAACATGGG
(SEQ ID NO: 34)
XA_23Rf: TGGCAGAAGCTGTTCCTGTCGAT
(SEQ ID NO: 35)
XA_24F: AGCTACGCCGAGATGTGGC
(SEQ ID NO: 36)
XA_25Rf: TGGATCTACGACGACTACTAC
(SEQ ID NO: 37)
XA_26F: GTCCGCGACGACGGCAAGACC
(SEQ ID NO: 38)
XA_27Rf: AAGCAGATCATGCGGTCCTAC
(SEQ ID NO: 39)
XA_28F: GTCCGCGACGACGGCAAGAC
(SEQ ID NO: 40)
XA_29F: TCCGCGGCAACATGTTCCG
(SEQ ID NO: 41)
XA_30F: GCGGTGCAGATGATCGACGA
(SEQ ID NO: 42)
XA_31Rf: GAGATGTGGCGGCGGTGGA
(SEQ ID NO: 43)
XA_32Rf: AACTACTGGCGGATCGACGCG
(SEQ ID NO: 44)
XA_33Rf: GACGGCAAGACCCTGGTC
(SEQ ID NO: 45)
Xmo_10F: TGGTGGAACAACCACTGCGTGGT
(SEQ ID NO: 46)
Xmo_11F: CAGTGGCGGACCTACTGCTCGG
(SEQ ID NO: 47)
Xmo_1F: TGGTTCGAGCACAACTAYCCNGGNTGG
(SEQ ID NO: 48)
Xmo_3Rf: AAGCCGATCGCGTTCGAGGA
(SEQ ID NO: 49)
Xmo_4F: GATACCAGTACCCGCACCG
(SEQ ID NO: 50)
Xmo_5Rf: CAGATGAACCTCAAGAAGCT
(SEQ ID NO: 51)
Xmo_6F: TACATGAACAACTACATCGA
(SEQ ID NO: 52)
Xmo_9F: CAGGAGGCGCACATTGAGTAGG
(SEQ ID NO: 53)
Xmo_F: ACGATCCAGATGAACCTCAAGA
(SEQ ID NO: 54)
Xmo_Rf: TACGCCGAGATGTGGCGGC
REVERSE PRIMERS:
(SEQ ID NO: 55)
XA_30Fr: ACCTCGTCGATCATCTGCA
(SEQ ID NO: 56)
XA_0288R: GACAACTCGGTGATCTTCGC
(SEQ ID NO: 57)
XA_0348R: GCCTTCTCGAAGGTGTAGTCGGT
(SEQ ID NO: 58)
XA_0360R: TCCTTCTTGGGGGCCTTCTCGAA
(SEQ ID NO: 59)
XA_0393R: CGGGAAGTAGGACCGCATGATCTG
(SEQ ID NO: 60)
XA_0408R: TTCTCTTCCTCCATCGGGAAGTA
(SEQ ID NO: 61)
XA_0444R: GGCACCGTCCATGGCGCCGTA
(SEQ ID NO: 62)
XA_0567R: ACCGCGTCGATGGCCATCGGCAT
(SEQ ID NO: 63)
XA_0624R: TGACGAACCTCGTCGATCATCTG
(SEQ ID NO: 64)
XA_0745R: CCGATGGTGCCCGCGTAGTTGTT
(SEQ ID NO: 65)
XA_0779R: GGTGATCGCGTCGCCGGTAATGAA
(SEQ ID NO: 66)
XA_0866R: TTGGCGGCCGCCTCGTCGGGCAT
(SEQ ID NO: 67)
XA_0944R: GAGTAGCCGTTGGAGATGTG
(SEQ ID NO: 68)
XA_0983R: AGTGGACGGTTGCGCTCGTCGGC
(SEQ ID NO: 69)
XA_1073R: TCCTTGGTGCCGTACTCGATGAA
(SEQ ID NO: 70)
XA_1091R: TCCCGGTCCTTGCGGCGGTCCTT
(SEQ ID NO: 71)
XA_1214R: CGCTTCCACGCCTCCTCGAC
(SEQ ID NO: 72)
XA_1327R: TGTGCTCGAACCACTCGAAGTCC
(SEQ ID NO: 73)
XA_1469R: GCGGGAACCATGCAGGTCCAGCA
(SEQ ID NO: 74)
XA_1548R: GTCCAGTAGCAGGTTTCCGAGCA
(SEQ ID NO: 75)
XA_1615R: CCCGTGAGCCGGCCCATGTTCGG
(SEQ ID NO: 76)
XA_1714R: TGACCGACCAGGGTCTTGCCGTC
(SEQ ID NO: 77)
XA_18Fr: CCGTTGGAGATGTGCCGCGA
(SEQ ID NO: 78)
XA_19Fr: TCGAACCACTCGAAGTCCG
(SEQ ID NO: 79)
XA_20R: CGAACGCGATCGGCTTGTT
(SEQ ID NO: 80)
XA_21R: TGAGCCGGCCCATGTTCGG
(SEQ ID NO: 81)
XA_22Fr: CCCATGTTCGGGGTCGGGC
(SEQ ID NO: 82)
XA_23R: ATCGACAGGAACAGCTTCTGCCA
(SEQ ID NO: 83)
XA_24Fr: GCCACATCTCGGCGTAGCT
(SEQ ID NO: 84)
XA_25R: GTAGTAGTCGTCGTAGATCCA
(SEQ ID NO: 85)
XA_26Fr: GGTCTTGCCGTCGTCGCGGAC
(SEQ ID NO: 86)
XA_27R: GTAGGACCGCATGATCTGCTT
(SEQ ID NO: 87)
XA_28Fr: GTCTTGCCGTCGTCGCGGAC
(SEQ ID NO: 88)
XA_29Fr: CGGAACATGTTGCCGCGGA
(SEQ ID NO: 89)
XA_30Fr: TCGTCGATCATCTGCACCGC
(SEQ ID NO: 90)
XA_31R: TCCACCGCCGCCACATCTC
(SEQ ID NO: 91)
XA_32R: CGCGTCGATCCGCCAGTAGTT
(SEQ ID NO: 92)
XA_33R: GACCAGGGTCTTGCCGTC
(SEQ ID NO: 93)
Xmo_10R: ACCACGAGTAGGTCCGCCACTG
(SEQ ID NO: 94)
Xmo_11R: CCGAGCAGTAGGTCCGCCACTG
(SEQ ID NO: 95)
Xmo_2R: TGCGGCTGCGCGATCAGCGTYTTNCCRTC
(SEQ ID NO: 96)
Xmo_3R: TCCTCGAACGCGATCGGCTT
(SEQ ID NO: 97)
Xmo_4Fr: CGGTGCGGGTACTGGTATC
(SEQ ID NO: 98)
Xmo_5R: AGCTTCTTGAGGTTCATCTG
(SEQ ID NO: 99)
Xmo_6Fr: TCGATGTAGTTGTTCATGTA
(SEQ ID NO: 100)
Xmo_Fr: TCTTGAGGTTCATCTGGATCGT
(SEQ ID NO: 101)
Xmo_R: GCCGCCACATCTCGGCGTA

4. An oligonucleotide complementary to the sequences of the gene prmD of propane-oxidizing bacteria according to claim 2, selected from the following sequences of forward and reverse primers for prmD:
FORWARD PRIMERS:

| | |
|---|---|
| XD_043F: TCGTCCACCGAGTTCTCCAACA | (SEQ ID NO: 102) |
| XD_071F: GTGTCACCTTGATGAACACCCC | (SEQ ID NO: 103) |
| XD_181F: AACCGGCTCGAGTTCGACTACG | (SEQ ID NO: 104) |
| XD_2Rf: GTTCTCCAACATGTGCGGCG | (SEQ ID NO: 105) |
| XD_3Rf: CCGTCGATGATCCGCGTC | (SEQ ID NO: 106) |
| XD_4Rf: TCTTCGAGGAGATCAGCTCCAC | (SEQ ID NO: 107) |
| XD_5Rf: GACGCCGCCGAGTACATCGG | (SEQ ID NO: 108) |
| Xmo_8F: ACCGAGTTCTCCAACATGTG | (SEQ ID NO: 109) |
| XD_6Rf: TTCGAGGAGATCAGCTCCACC | (SEQ ID NO: 110) |
| Xmo_7Rf: CATGCAATTCGGATCGKCCA | (SEQ ID NO: 111) |
| XD_7F: GGCTCCATCTTCGAGGAGATCA | (SEQ ID NO: 112) |

REVERSE PRIMERS:

| | |
|---|---|
| prmD_1R: ATGGACCATCCGNCCRTARTGNGT | (SEQ ID NO: 113) |
| XD_061R: ACGCGGCCGATCGGGGTGTTCAT | (SEQ ID NO: 114) |
| XD_136R: TGGCCGTCGACGCGGATCATCGA | (SEQ ID NO: 115) |
| XD_172R: TCGGTGAGCTCGTCGTAGTCGAA | (SEQ ID NO: 116) |
| XD_235R: TGGGTGGAGCTGATCTCCTCGAA | (SEQ ID NO: 117) |
| XD_2R: CGCCGCACATGTTGGAGAAC | (SEQ ID NO: 118) |
| XD_3R: GACGCGGATCATCGACGG | (SEQ ID NO: 119) |
| XD_4R: GTGGAGCTGATCTCCTCGAAGA | (SEQ ID NO: 120) |
| XD_5R: CCGATGTACTCGGCGGCGTC | (SEQ ID NO: 121) |
| XD_6R: GGTGGAGCTGATCTCCTCGAA | (SEQ ID NO: 122) |
| XD_7Fr: TGATCTCCTCGAAGATGGAGCC | (SEQ ID NO: 123) |
| Xmo_7R: TGGMCGATCCGAATTGCATG | (SEQ ID NO: 124) |
| Xmo_8Fr: CACATGTTGGAGAACTCGGT. | (SEQ ID NO: 125) |

5. A pair of oligonucleotides complementary to the sequences of the gene prmA of propane-oxidizing bacteria according to claim 1, comprising a forward oligonucleotide and a reverse nucleotide selected from the sequences of claim 3.

6. A pair of oligonucleotides according to claim 5, selected from the following pairs of sequences:

| | |
|---|---|
| XA_16F: GGCGCACATTGAGTAGGCA | (SEQ ID NO: 27) |
| XA_23R: ATCGACAGGAACAGCTTCTGCCA | (SEQ ID NO: 82) |
| XA_16F: GGCGCACATTGAGTAGGCA | (SEQ ID NO: 27) |
| Xmo_5R: AGCTTCTTGAGGTTCATCTG | (SEQ ID NO: 98) |
| XA_19F CGGACTTCGAGTGGTTCGA | (SEQ ID NO: 30) |
| XA_21R TGAGCCGGCCCATGTTCGG | (SEQ ID NO: 80) |

7. A pair of oligonucleotides complementary to the sequences of the gene prmD of propane-oxidizing bacteria according to claim 2, comprising a forward oligonucleotide and a reverse nucleotide selected from the sequences of claim 4.

8. A pair of oligonucleotides according to claim 7, selected from the following pairs of sequences:

| | |
|---|---|
| Xmo_8F: ACCGAGTTCTCCAACATGTG | (SEQ ID NO: 109) |
| XD_5R: CCGATGTACTCGGCGGCGTC | (SEQ ID NO: 121) |
| Xmo_8F: ACCGAGTTCTCCAACATGTG | (SEQ ID NO: 109) |
| prmD_1R: ATGGACCATCCGNCCRTARTGNGT. | (SEQ ID NO: 113) |

9. A method for the identification of propane-oxidizing bacteria comprising the extraction of DNA from environmental samples and the subsequent identification of at least one fragment of the gene prmA according to the prmA sequences of claim 1, and/or of the gene prmD according to the prmD sequences of claim 2, characterized in that the identification of said gene fragments is carried out by gene amplification in the presence of pairs of primers selected in correspondence of homologous portions deduced from the alignment of the prmA and prmD sequences according to claims 1 and 2.

10. The method according to claim 9, wherein the identification of the prmA gene is carried out by means of gene amplification in the presence of pairs of forward and reverse primers indicated in claims 5 and 6.

11. The method according to claim 9, wherein the identification of the prmD gene is carried out by means of gene amplification in the presence of pairs of forward and reverse primers indicated in claims 7 and 8.

12. A method for the identification of propane-oxidizing bacteria comprising the hybridization of a suitably labelled probe with the DNA of the sample to be analyzed, characterized in that the probe consists of at least one of the sequences indicated in claims 3 and 4.

13. The method according to claim 12, wherein the DNA consists of the product of gene amplification of claim 9.

14. A method for the identification of propane-oxidizing bacteria according to claim 9 comprising the following steps:
extracting the DNA from samples;
putting the extracted DNA in contact with a pair of primers complementary to the prmA or prmD gene under conditions which allow the amplification of a fragment of the prmA or prmD gene;
analyzing the gene amplification product by means of real time PCR, gel-electrophoresis or another analysis method.

15. A method for the quantitative determination of propane-oxidizing bacteria, comprising:
performing gene amplification according to the method of claim 14 in the presence of different quantities of genomic DNA of propane-oxidizing bacteria;
quantitative determination of the gene amplification product;
construction of a calibration curve;
quantitative determination of the genomic DNA in samples to be analyzed by means of interpolation.

16. A Kit for the identification of the presence of propane-oxidizing bacteria in environmental samples or of other types, based on the identification of prmA and/or prmD genes according to the method of claim 9.

17. Use of the sequences of prmA and prmD genes according to claims 1 and 2 for the identification of primers for gene amplification.

18. A method for discovering the presence of oil or natural gas reservoirs, based on the identification of propane-oxidizing bacteria according to the method of claim 9.
